(19) United States
(12) Patent Application Publication
(10) Pub. No.: US 2002/0160428 A1
(43) Pub. Date: Oct. 31, 2002

(54) Quantitative non-instrumental immunoassay and device using coloured particles

(76) Inventor: Erling Sundrehagen, Oslo (NO)

Correspondence Address: MERCHANT & GOULD PC, P.O. BOX 2903, MINNEAPOLIS, MN, 55402-0903, US

(21) Appl. No.: 10/132,989

(22) Filed: Apr. 26, 2002

(30) Foreign Application Priority Data
Apr. 30, 2001 (NO) 20012150

(51) Int. Cl. G01N 33/53; G01N 33/542; C12M 1/34

(52) U.S. Cl. 435/7.9; 435/287.2

(57) ABSTRACT

The present invention provides a reproducible method of analysis for determining the concentration of one or several analytes in a sample, a device for performing the method, use of the method to perform specific analysis and a kit for performing the method.

The present invention relates to a method for determining the concentration of one or several analytes in a sample, a device for performing the method, use of the method to perform specific analysis and a kit for performing the method.

In the field of analytic biological chemistry there is a considerable need of methods for rapid qualitative and quantitative determination of analytes in biological fluids, requiring as few analytical steps as possible, and no special skills on behalf of the person performing the analysis.

In 1986 Mochnal & al. of Ortho Diagnostic Systems within the Johnson & Johnson Corporation filed a patent application, later granted as European Patent 0250137 B1. The invention provided a method for quantification of an analyte (i.e. the substances to be determined) in a complex test sample, e.g. urine or blood, characterized by having a porous membrane strip with immobilized binding molecules, and using other molecules coated on gold colloid particles that are brought into contact with an aliquot of the test sample which is allowed to stream through the porous membrane strip. The length of the part of the strip where said gold colloid particles are retained, is proportional to the concentration of the analyte molecules in the test sample.

This method was exemplified by a method description for quantitative analysis of luteinizing hormones in urine and for human gonadotrophine in urine and the medical substance theophyllin in blood, for which the length of the stripe created by the signal-providing gold colloid particles in the prescribed test strip was directly proportional to the concentration of analyte molecules in the test sample. The reagents were simple and quick and have a low production cost, and required no signal developing reagents, special equipment or temperature control. Still, neither Johnson & Johnson nor Ortho Diagnostic Systems have ever launched commercial products based on this technology.

EP 0250137B1 offers no detailed description of how element C of claim 1 should be carried out, that is; no detailed description of the transfer of fluid to the membrane strip. At the bottom of page 3 it is indicated that the reagents are held in a container and that the reagents are brought into contact with the said membrane strip, containing immobilized binding molecules. The only exemplification of carrying out element C of claim 1 is found in the first line on page 5, where it is described that one end of the membrane strip is dipped into a mixture of test sample and reagent. Similarly there is a description, in the first line on page 10 in example 7, of the strip being immersed to a depth of 10 mm. In example 3, description of the membrane, no detail is offered beyond a thorough description of the membrane strip. Consequently, the only reasonable interpretation is that this membrane strip is dipped into reagent/test sample without any particular transfer devices.

There are considerable disadvantages connected with such a dipping technology when it is attempted made quantitative. One disadvantage is that precise preparation of the reagent/test sample mixture might require practice and/or competence in precise preparation of the mixture. Since the membrane strip is to be held down in the reagent/test sample, either a holding device has to be made for the membrane strip or one must use a test tube to hold the membrane strip, which could function as a container for the reagent/test sample as well. In such tubes fluids tend to migrate differently along the edges of the membrane strip than in the center of the strip, and the liquid front easily becomes uneven. The strip described in EP 0250137 B1 could, however, be well suited for quantitative analyses of analytes with relatively little biological variation, or for instance medical substances with so-called narrow therapeutic width (small difference in concentration in blood between therapeutic and toxic values).

The only commercial product, known to the present inventor, that puts to use anything resembling the principle of EP 0250137 B1 is the firm Syva's (Later Dade Behring, one, of the world's largest diagnostics products firms) immunochromatographic enzyme-based product, described in U.S. Pat. No. 443504, which used tube-shaped containers and narrow membrane strips. The product is described in the article >>Enzyme immunochromatography—a quantitative immunoassay requiring no instrumentation>>, in Clinical Chemistry vol. 31, 1144-1150, 1985. But Syva's product made use of several reagent containers, including enzyme substrate containers, and the method was quite complicated to carry out. Despite great demand the product was discontinued, according to Syva's sales representative in Norway, due to a complicated and costly industrial production. EP 0250137 B1 describes strips containing different quantities of specific binding molecules per unit of area in different sections of the strip, to measure wider ranges of concentration (also called larger dynamic measurement range). The patent holder has, however, never marketed such strips, and it is technically and industrially complicated to implement such production with good precision. A simpler approach is to make use of radial analysis techniques as introduced by Mancini et al., Immunochemistry, 2: 235-254 (1965)) in radial immunoprecipitation techniques in agarose gel. Using radial migration a significantly larger dynamic measurement range can be achieved, since the migration length in this format becomes proportionate to the square root of the area. Increasing the radius from 1 to 3 cm will thus result in an area increase by factor 9, and will thus be applicable in a larger concentration measurement range.

At the time of Ortho's and Sylva's commercially less successful development of the principles for area measurement in immunochromatography, a commercially very successful development of another main principle for thin-layer immunochromatography also took place, to most people known from modern pregnancy tests, and reference can be made to e.g. Rosenstein & Bloomster's U.S. Pat. No. 4,855,240 and May & al. in EP 291 194, 1988. It is characteristic of this technology that the test sample, with or without added reagent, is dripped into a well or onto a piece of filter affixed to a moisture absorbing membrane strip, whereby the test sample migrates into and further along the porous membrane. The migrating liquid dissolves desiccated specific binding molecules that have previously been chemically bound to signal-providing substances, and these binding molecules (typically antibodies) in turn bind to the analyte molecules from the test sample. Further along in the migration strip some more specific binding molecules have been immobilized, typically in a stripe perpendicular to the migration direction or for instance in a pattern, e.g. a cross. When the analyte molecules carrying specific binding molecules, which in their turn have signal providing substances attached to them, pass the said stripes or patterns containing immobilized binding molecules, the signal-providing substances are concentrated in these stripes or patterns. A positive test is read as color or fluorescence in the given stripe or pattern. A large number of firms produce and market such products.

Starting with two out of the world's three largest diagnostics firms and their lists of products, we see that this form of qualitative thin-layer immunochromatography is used extensively. Bayer, USA, sells Clinitek hCG urine test and Clinitek Microalbumin urine test. Abbott Laboratories in the US sells Fact Plus Pregnancy Test, TestPack hCG Combo, TestPack Chlamydia, TestPack Strep A, TestPack Rotavirus, and TestPack RSV. Of the smaller but more specialized firms we might mention Nulbenco Medical International, USA, which sells these types of tests for hCG, LH, Chagas, Chlamydia, Cholera, CK MB, Dengue, Myoglobin, Strep A, Hepatitis B antigen, Tropnin I, Hemoglobin in stool, as well as for antibodies for Deng, Helicobacter Pylori, Hepatitis B antibodies, mononucleosis antibodies, antibodies for Treponoma Pallidum and Mycobacteria Tuberculosis, and also for detection of the tumor markers Alpha Fetoprotein, Carcinoembryonal antigen and Prostatospesific antigen. Millipore Inc in the US, which is a specialized producer of filter materials, sells full hardware <<assembly kits>> for these types of tests from their OEM-department, so-called HiFlow assembly kits. Pall Gelman plc, UK, has even issued a manual for manufacturing such products, viz. their brochure <<Immunokniatographic, Lateral Flow or Test Strip Development Ideas>> which can also be downloaded from their Internet site. Acon Laboratories, Inc., in the US, has a big own production and sales of these types of tests, especially to the Chinese market, but also deliver tests, so-called OEM, to other firms for resale under the customer's trademark.

Apparatuses for measuring the intensity of these stripes or patterns have also been constructed, but it has been difficult to design chemicals and devices providing sufficiently precise and accurate results. Highly sophisticated technologies have been developed to overcome these limitations, viz. typically U.S. Pat. No. 6,136,610: <<Method and apparatus for performing a lateral flow assay>> by Polito & al. It is evident from U.S. Pat. No. 6,136,610 that more complicated and advanced methods and apparatuses are needed to make this method quantitative, and it is very complicated and costly to achieve industrially reproducible precision and accuracy, until now it actually hasn't been feasible in a commercial context.

Roche Diagnostics has developed a variety of this chromatographical principle in which the intensity of coloration in the testing section to which the signa-providing substances proceed is used as a semi-quantitative measure for determining albumin in urine. Diabetes care, vol. 20, number 11, pp. 1642-1646, describes this.

U.S. Pat. No. 5,958,790 by Erich Cerny describes a vertical filter immunoassay method based on vertical flow of sample aliquot through a filter with specific binding molecules, followed by binding molecules with attached signal-providing substances such as gold colloids. The method is used commercially by reading the intensity of the light reflection using a reflectometer in its quantitative embodiment, and the method requires accurate pipetting of reagents. Using volume calibrated pipettes and a reflectometer the method can be made quantitative with good precision.

Hajizadeh and Wiljesuriya in U.S. Pat. No. 6,180,417 and EP 1 046 913 A2 describe an immunochromatographic strip that has a non-porous receiving unit, which is in direct contact with the absorbing material in the chromatography strip. It remains to be seen whether Bayer will be able to solve the industrial problems that have been limiting manufacturing of these types of industrial products, and it is remarkable that Bayer only describes application of their device in connection with qualitative analysis products.

Since the described lateral or vertical immunochromatographic methods have been impossible to perform quantitatively without using instruments, then why haven't Ortho or, later on, Johnson & Johnson or Syva or, later on, Dade Behring developed further their lateral thin-layer chromatographical methods described in EP 0250137 B 1 and U.S. Pat. No. 4,435,504 for commercial quantitative analysis products using such wells or affixed pieces of filter for drip application of reagents? The present inventor and many of my colleagues have tried to construct wells or affixed pieces of filter providing regular and reproducible migration patterns and areas in accordance with the principles of EP 0250137 B1, without succeeding. Border effects and contact effects have made it impossible to create a reproducible solution on an industrial scale. Various linings, o-rings and different types of glue or adhesives have been tried unsuccessfully. Thus, there still exists a need for an industrially reproducible method of analysis in which signal-providing substances migrate reproducibly and provide patterns or areas the size of which can be applied directly to determine the concentration of one or several analytes in a test sample with a large dynamic concentration measurement range, suitable for being performed by persons without specialized laboratory training.

It is therefore an object of the present invention to provide a method for determining the concentration of one or several analytes in a sample, a device for performing the method, use of the method to perform specific analysis and a kit for performing the method. These objects have been obtained by the present invention, characterized by the enclosed claims.

The present invention relates to a quantitative chemical method of analysis for determining concentrations of one or several analytes in a sample, wherein a sample containing the analyte or analytes is mixed with a reagent contained in a container, wherein the reagent contains signal-providing substance(s), thus providing a mixture which is subsequently absorbed by a fluid-transmitting material contained in a fluid-transmitting device after coupling of the container to the fluid-transmitting device, and simultaneously or afterwards bringing the fluid-transmitting device in contact with a fluid-receiving device containing a fluid-receiving material which includes immobilized reagents with specific binding capacity for the analyte or analytes, or immobilized analyte molecules or analogues or derivates or fragments thereof, wherein the mixture is transported out in the porous fluid-receiving material in the said other fluid-receiving device and create a pattern wherein the pattern or area of the pattern or area of the pattern elements are utilized as a measure of the concentration of analyte or analytes in the sample.

More specific the present invention relates to a quantitative chemical method of analysis for determining concentrations of one or several analytes in a test sample wherein the sample is mixed with the reagent, such as a liquid reagent in a container containing signal-providing substances,

a fluid-transmitting device containing a fluid-transmitting material is introduced into the said container so that the said fluid-transmitting material comes into contact with the said mixture in the said container,

the said fluid-transmitting material in the said fluid-transmitting device in the course of performing the said chemical method of analysis is brought into simultaneous contact with on the one hand the said mixture of reagent and test sample and on the other hand into contact with a porous fluid-receiving material in another fluid-receiving device, wherein the said fluid-transmitting material in the said fluid-transmitting device is not permanently mounted in contact with the porous fluid-receiving material in the said fluid-receiving device, but is brought into such contact as a part of performing this method, and wherein the said porous fluid-receiving material in the said other fluid-receiving device includes immobilized reagents which have specific binding affinity for the said analyte or analytes or that the said immobilized reagents consist of immobilized analyte molecules or analogues or derivatives or fragments of analyte molecules, whereby the said mixture is transported through the fluid-transmitting device and over into and spreads out in the porous fluid-receiving material in the said other fluid-receiving device, whereby the pattern, the area of the pattern and/or the area of the pattern elements that emerge through the distribution of the signal-providing substances in the said porous fluid-receiving material in the said fluid-receiving device, are utilized as a measure of the concentration of analyte or analytes in the sample.

The contact between the said fluid-transmitting material in the said fluid-transmitting device with on the one hand the mixture of reagent and test sample in the said container and on the other hand with a porous fluid-receiving material in the said other fluid-receiving device) can comprise a contact which is established either simultaneously, or first with the mixture of reagent and test sample, or first with the porous fluid-receiving material in the said other fluid-receiving device.

An especially preferred embodiment of the present invention is characterized by the said container being a liquid leak proof container, and further characterized by the fluid-transmitting device, which contains a fluid-transmitting material, being led through a liquid leak proof gate into the said container in such a way that the said fluid-transmitting material comes into contact with the said mixture in the said container.

It is further characteristic of the present invention that the fluid-transporting material in the fluid-transmitting device can consist of a porous fluid-transporting material suitable for transporting fluids using capillary forces or overpressure or underpressure.

Another embodiment of the present invention is characterized by the inclusion in the fluid transmission device of a non-porous nib or a tube-shaped transmission which is not mounted in permanent contact with the fluid-receiving device, but which is brought into contact with the fluid-receiving device during the process of carrying out the quantitative chemical method of analysis.

What further characterizes the method related to the present invention is that the said container for mixing of reagent with test sample can be a closed container with a gate at which the said fluid-transmitting device can come into contact with the said mixture; if expedient, by supplying the said container with a notch in a wall where the wall is thinner and yields when the transmission device is led through in a tight fitting manner or; if expedient, by the fluid-transmitting unit and the said container being screwed together, if expedient with small gas permeable openings in the container or transmission device, shaped in such a manner that the said mixture does not leak out of the container or fluid transmission device regardless of the spatial position in which the container and/or fluid transmission device are/is held.

What further characterizes the present invention is that the said container for mixing of reagent and test sample can be equipped with a gate for introduction of test sample or that a third device containing the test sample is used and, if desirable, that the said third device constitutes a part of the said container when it is joined together with or screwed onto the other devices. Furthermore the said third device is not a part of the container and is e.g. a glass capillary.

What further characterizes the present invention is that the said fluid-receiving device contains specific binding molecules with affinity for analytes or the analytes, or for analogues of or derivatives of or fragments of or whole analyte molecules, either in immobilized form and/or in desiccated form or dispersed onto or into particles or directly into the porous fluid-receiving material in the said fluid-receiving device, with a homogeneous or inhomogeneous—but previously determined—distribution in the porous fluid-receiving material.

What further characterizes the present invention is that the said reagent can contain signal-providing substances in the form of colored particles or colloids or enzymes or fluorophores or dyes, with or without attached specific binding molecules or with or without attached analogues of or derivatives of or fragments of or whole analyte molecules.

What further characterizes the present invention is that the said reagent can include chemicals that dissolve cells in the test sample and/or regulate the acidity or ionic strength or keep any possible particles dispersed.

Furthermore, what characterizes the present invention is that the said fluid-transmitting device can have a pore size that holds back cells such as red or white blood cells, but has a pore size large enough to let through the said signal-providing substances.

What further characterizes the present invention is that the hemoglobin in the test sample can be used as signal-providing substance.

What further characterizes the present invention is that the test sample can be pretreated by adding chemicals or be separated or extracted prior to being mixed with the said reagent or that the said reagent can be provided by mixing together two or several different reagents inside the said container, or that additional chemicals are added to the porous fluid-receiving material in the fluid-receiving device in order to evoke or enhance or clarify the patterns or areas of patterns and/or the area of the pattern elements that appear in the said fluid-receiving device.

A characteristic of the present invention is that the patterns or areas of patterns and/or the area of pattern elements that appear in the said fluid-receiving device can be depicted or scanned or measured using analogue or digital instruments based on visible or ultraviolet or infrared or near-infrared light, either by absorption measurement or reflection measurement or fluorescence measurement, and that the concentration of the analyte or the analytes in the test sample is determined on the basis of these measurements.

A distinct embodiment of the present invention is further characterized by the use of a leak proof container for the mixture of reagent and test sample, and further characterized by the fact that the fluid-transmitting device contains a porous fluid-transmitting material which is mounted in permanent contact with the porous fluid-receiving material in the fluid-receiving device.

The present invention relates also to a device for performing a method for determining concentrations of one or several analytes in a test sample, comprising a liquid leak proof container for mixing of the test sample with a reagent, a fluid-transmitting device which contains a fluid-transmitting material, and a fluid-receiving device which contains a fluid-receiving material, assembled such that the fluid-transmitting device is able to be contacted with the content of the said container through a liquid leak proof port and contacted with the fluid-receiving device containing the fluid-receiving material.

Further the invention relates to a device in wherein the fluid-transporting material in the fluid-transmitting device consists of a porous fluid-transporting material suitable for transporting fluids using capillary forces or overpressure or underpressure.

The invention also relates to a device in wherein a non-porous nib or a tube-shaped transmission is included in the fluid transmission device, not mounted in permanent contact with the fluid-receiving device, but brought into contact with the fluid-receiving device during the process of carrying out the quantitative chemical method of analysis.

In a further embodiment the device in accordance with the present invention the said leak proof container has a port through which the said fluid-transmitting device can come into contact with the said mixture of test sample and reagent, suitably that the said container has a notch in a wall where the wall is thinner and yields when the transmission device is led through in a tight fitting manner or the fluid-transmitting unit, and the said container being screwed together, suitably with small gas-permeable openings in the container or transmission, shaped in such a manner that the said mixture does not leak out of the container or fluid transmission device regardless of the spatial position in which the container with the fluid transmission device is held.

Furthermore the device in accordance with the present invention is characterized in that the said container for mixing of reagent and test sample is equipped with a port for introduction of the test sample from a sample transporting device, such as e.g. a glass capillary, or that the sample transporting device constitutes a part of the said container, such as a lid device which is joined together with, or screwed onto the said container in the port location.

According to the present invention the device is characterized by the said fluid-receiving device containing specific binding molecules with affinity for analytes or the analytes, or for analogues of or derivatives of or fragments of or whole analyte molecules, either in immobilized form and/or in desiccated form or dispersed onto or into particles or directly into the porous fluid-receiving material in the said fluid-receiving device, with a homogeneous or inhomogeneous, but previously determined, distribution in the porous fluid-receiving material.

Furthermore the device in accordance with the present invention is characterized in that the said container contained reagent comprises signal-providing substances in the form of colored particles or colloids or enzymes or fluorophores or dyes, with or without attached specific binding molecules or with or without attached analogues of or derivatives of or fragments of or whole analyte molecules.

In a further embodiment the device in accordance with the present invention is characterized in that the said reagent includes chemicals that dissolve cells in the test sample and/or regulate the acidity or ionic strength or keep any possible particles dispersed.

In a still further embodiment the device in accordance with the present invention is characterized in that the said fluid-transmitting material in the said fluid-transmitting device has a pore size that holds back cells, such as red or white blood cells, but with a pore size large enough to let through the said signal-providing substances.

In a further embodiment the device in accordance with the present invention is characterized in comprising a stopper (6) with a built in capillary (7), a sealing sleeve (8) surrounding the stopper, a liquid leak proof container (9), a movable ball (10) sealing the port in the bottom of the container (9), wherein the ball (10) is housed in a valve seat(11) which is sealingly fitted to a wick or felt tip guide (12), wherein a wick or felt tip (13) is sealingly and movable mounted, wherein the felt tip (13) is protected by a removable cap (14)

In a still further embodiment the device in accordance with the present invention is characterized by further comprising a scanning device, such as analogue or digital instrument based on visible or ultraviolet or infrared or near infrared light, or a combination thereof, to measure absorption or reflection or fluorescence, or a combination thereof, a processor for processing the data, a display medium, and medium for storing the data.

In a further embodiment the device in accordance with the present invention is characterized by further comprising a rack with a movable holder, whereby the container is fixed in a standardized position in relation to the fluid-receiving device such that only vertical controlled movement is possible.

A further embodiment of the present invention relates to use of the method wherein the concentration of one or several analytes in a biological sample, such as blood, sputum, mucus, faeces, expectorates and tissue is measured.

In a further use of the method according to the present invention the analytes are selected from the group comprising autoantibodies, antibodies, saprophytes, bacteria, other infectious agents, hemoglobin, albumin, CRP, U-albumin, glycated albumin, glycated hemoglobin, ferritin, ASAT, ALAT, LDH, myoglobin, Troponin I, Fatty Acid Binding Protein, amylase, HCG, U-HCG, theophyllin, and antibiotics.

The present invention also relates to a kit for performing the method comprising the said device, reagent for mixing with the test sample, optionally additional reagents for pretreatment or separation of the test sample or admixing into the fluid-receiving device for clarification of the signal.

The present invention will now be described in more detail, with reference to figures and examples.

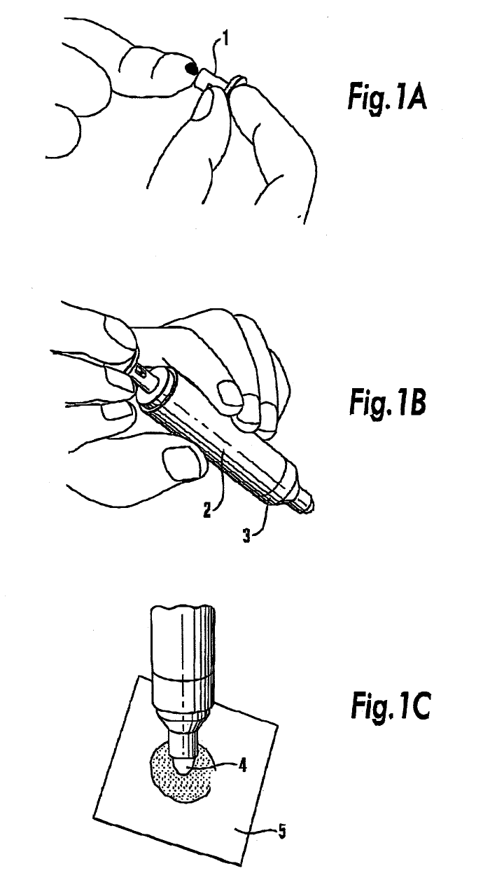

FIG. 1A, B and C illustrates one embodiment of the device for mixing and transmitting the test sample-reagent mixture and the fluid-receiving device.

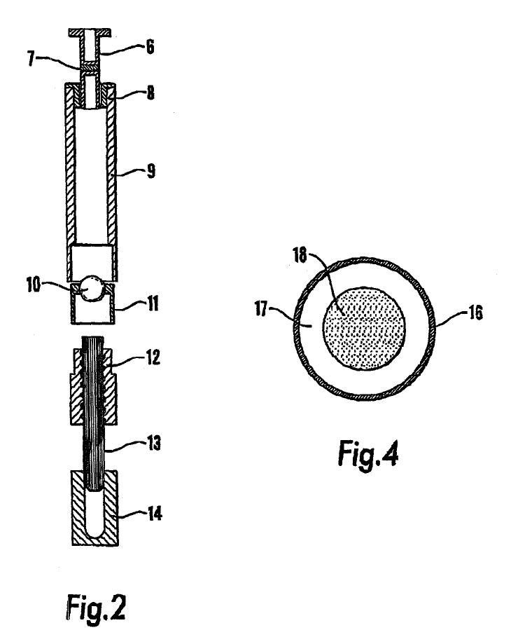

FIG. 2 illustrates a second embodiment of the device for mixing and transmitting the test sample-reagent mixture.

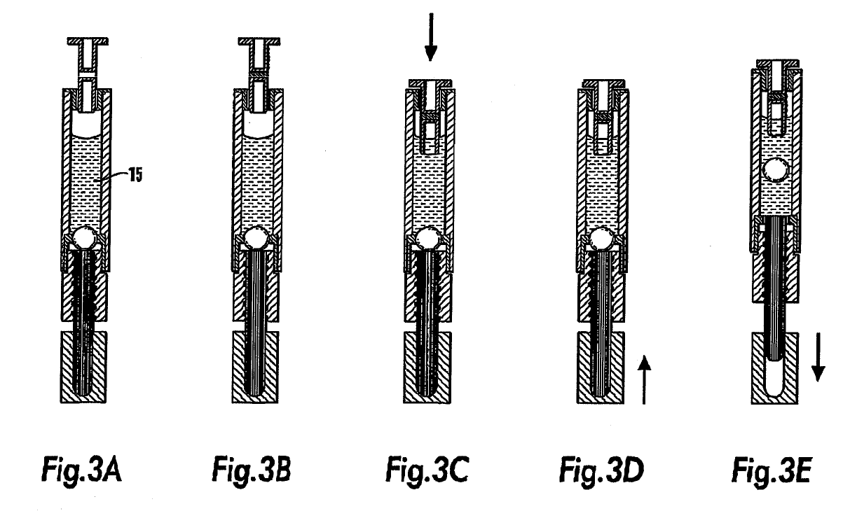

FIG. 3A, B, C, D and E illustrates the use of the embodiment illustrated in FIG. 2.

FIG. 4 illustrates an embodiment of the fluid-receiving device.

The present invention provides a method and device for quantification of one or several analytes in a test sample or in test sample material using one single liquid reagent. This reagent includes signal-providing substance(s) and is used in combination with a container for mixing a sample 2,9-readily an aliquot of a test sample—into the reagent 15 thus providing a mixture, a fluid transmission device 4,13, and a fluid-receiving device 5,16 including a porous material 17 which receives the transmitted fluid (i.e. fluid-receiving material).

According to the present invention, it has also been possible to make a comprehensive device, which can be characterized as a pen for quantification of analytes in complex test samples. Viewed from the exterior in its most preferred embodiments it resembles a felt tip pen or a fountain pen or cartridge pen with a felt tip or fiber tip 4, 13 or nib, whereby fluid from a container is led through a transmission device down onto a two-dimensional matrix 5,17 made out of e.g. paper or a filter material, for instance nitrocellulose or more modern further developed materials with similar properties. The device further contains a stopper 1, 6, with a built-in capillary 7, and a felt tip guide 3,12 which holds the felt tip 4, 13.

Suitable materials for quantitative analysis in compliance with the present invention (i.e. use of the invention) are body fluids or extracts thereof, typically urine, saliva, blood serum, blood plasma, blood hemolysate, anti-coagulated blood or full blood, cerebrospinal fluid, extracts or fractions of body fluids, or fluids or extracts from the plant kingdom, or fluids or suspensions or other liquid or suspended states of aggregation in nature, such as aqueous solutions, e.g. waste water. In the present invention a sample—readily as an aliquot of a sample material—is mixed with a reagent in a. container. The aliquot is led into the container after having been sucked up into a sample-taking device, e.g. a small capillary tube with a predetermined internal volume, which is filled as a result of the test sample displacing the air inside the capillary tube, due to the surface tension. This capillary tube can be of any suitable form, such as e.g. straight or spiral shaped or be inside a device which can also serve e.g. as a stopper in the said container, so that the device closes the container in such a way that it becomes liquid leak proof when and after the test sample is mixed with the reagent. This mixture can for instance come about through shaking of the container, manually or using an instrument, so that the test sample flows out into the reagent and mixes.

A liquid leak proof container for the mixture of reagent and test sample is a preferred embodiment, but not required. The advantage is that the container in the liquid leak proof embodiment can be held in all possible spatial positions without the liquid mixture leaking out, in the same way as with a pen, which it should preferably be possible to hold in different positions when producing the desired writing.

A further characteristic of the invention is that a fluid-transmitting device is led into the said container, preferably through a leak proof gate. This fluid transmission device can in a preferred embodiment consist partly or completely of a porous material which absorbs liquids, analogous to the tip of felt tip pens or cartridge pens or India ink pens, or in other design varieties in the form of a tube-shaped material or a wick, or a split-shaped device, such as the nib of a pen or a thin metal tube. The reagent container and the said transmission device can thus be designed similarly to a fountain pen or an India ink pen, in which the reagent/test sample mixture is transferred via a nib or a tube-shaped transmission or preferably a transmission consisting of a porous material that absorbs aqueous liquids, but which differs somewhat from the most common pens in that the transmission device should not be put into contact with the reagent until after the reagent is mixed with the test sample aliquot. This can for instance be achieved by providing the said container with a notch in one wall, at which point the wall is thinner and yields when the fluid transmission device is led through in a closefitting manner, or by screwing together the fluid-transmitting unit and the said container, or by providing the fluid-transmitting unit with a hollow tip which is pressed into the said container.

In a liquid leak proof embodiment it will, as a rule, be necessary to let air into the container to avoid underpressure in the container which would restrain the fluid transmission when fluids migrate out of the container through the fluid transmission device in order to avoid underpressure in the container which would restrain the fluid transmission. Small gas-permeable openings can therefore be used with advantage. It is advantageous to use openings that are so small or narrow that the fluid due to surface tension does not leak out, but big enough to allow gas molecules to diffuse in.

The present invention further uses a fluid-transmitting device containing a fluid-transmitting material which is led into the said container in such a way that the said fluid-transmitting material comes into contact with the said mixture in the said container. Further, the said fluid-transmitting material in the said fluid-transmitting device is brought into contact with on the one hand the said mixture of reagent and test sample and on the other hand into contact with a porous fluid-receiving material in another fluid-receiving device, either simultaneously or that the fluid-receiving material is contacted with the fluid transmitting material afterwards. The invention is further characterized by the said fluid-transmitting material in the said fluid-transmitting device not being permanently mounted in contact with the porous fluid-receiving material in the said other fluid-receiving device, but is brought into such contact as a part of applying this method.

A particularly preferred embodiment of the present invention is characterized by the use of a porous fluid-transmitting material in the said fluid-transmitting device. This porous fluid-transmitting material is simultaneously in contact with the mixture of reagent and test sample in the said container and in contact with a porous fluid-receiving material in a separate fluid-receiving device. The fluid mixture will thereby be transported through the transmission device and into the porous fluid receiving material in the fluid-receiving device. By using a fluid-transmitting device which is not permanently mounted onto or in contact with the porous fluid-receiving material in the fluid-receiving device, one can achieve an industrially producible device for a reproducible method for transmission of fluids with good and regular dissemination patterns in the porous material in the said fluid-receiving device. The fluid will thus spread out evenly and regularly in the porous fluid-receiving material in the fluid-receiving device. This is illustrated in FIGS. 1-4.

FIG. 1a illustrates blood sample taking in a sample-taking device with built-in capillary, FIG. 1b illustrates introduction of test sample into container 2 already containing the reagent characteristic of the present invention, FIG. 1c illustrates transmission of the mixture of reagent and test sample from the said container 2 through the fluid transmission device and into the porous fluid-receiving material in the fluid-receiving device 5, and a fluid dissemination pattern in the said porous fluid-receiving material in the fluid-receiving device. The pattern or the area of the pattern or pattern elements that appear as a result of the signal-providing substances' dissemination in the porous material in the fluid-receiving device can thereby be used as a measure of the concentration of analytes or the analytes in the test sample, according to the same principles that are described in EP 0252137 B1, but not limited to the type of signal-providing substances described in EP 0250137 B1.

In FIG. 2 another embodiment of the device is described, comprising a stopper 6 with a built-in capillary 7, such as e.g. a capillary holding 5 μl fluid, a sealing sleeve 8, a liquid proof container 9, a ball 10 sealing the port in the bottom of the container 9 wherein the ball is housed in a valve seat 11 which is formed to receive a wick or felt tip guide 12 in a sealing connection. The wick or felt tip 13 in a sealing and sliding connection in the wick or felt tip guide 12, and the tip is protected by a cap 14. All parts of the device is produced by suitable materials such as e.g. plastic, except the wick or felt tip which is made of a fluid-transmitting material.

FIG. 3 illustrates the use oh the embodiment illustrated in FIG. 2. FIG. 3 A depicts a device filled with the reagent 15, e.g. such as it may be provided commercially. The fluid-transmitting device is not in contact with the reagent 15 and the felt tip 13 is protected by the cap 14. In FIG. 3B the capillary 7 is filled with a test sample, e.g. blood and in FIG. 3C the stopper 6 is pressed downwards in order to bring the content of the capillary 7 into contact with the reagent 15. In FIG. 3D the reagent 15 is mixed with the test sample by moving or stirring the device. In FIG. 3E the fluid-transmitting device 13 is pushed against the ball 10 which then moves into the container 9 and contact is established between the fluid-transmitting device and the mixture of the test sample and the reagent 15.

FIG. 4 illustrates an embodiment of the fluid-receiving device comprising a circular tray 16 made of suitable materials such as e.g. plastic, wherein the fluid-receiving material 17 is located. The grey area 18 illustrates the pattern appeared as a result of the signal providing substances' dissemination in the porous material 17.

Particularly preferable is an evenly thick fluid-receiving porous material in the fluid-receiving device with evenly immobilized dissemination of immobilized reagents of specific binding affinity for the said analyte or analytes or that the said immobilized reagents consist of immobilized analyte molecules or analogues or derivatives or fragments of analyte molecules, because this will provide direct proportionality between the analyte concentration in the test sample and the area of the dissemination patterns that the signal-providing substances form in the porous fluid-receiving material in the fluid-receiving device.

To obtain the largest possible dynamic measurement range for the method the said porous fluid-receiving material in the fluid-receiving device will preferably be circular, and the said fluid transmission device will be brought into contact with the center of the porous fluid-receiving material in the said fluid-receiving device, and rings will be formed by the signal-providing substances. If the container for the mixture of reagent and test sample together with the device for fluid transmission have a pen-like design, the tip of this pen could typically be placed in the center of the porous fluid-receiving material in the fluid-receiving device, and rings will be formed by the signal-providing substances, and these rings can be measured using simple means.

If the said reagent includes immobilized specific binding molecules with affinity for the analyte molecules, the said analyte molecules with attached signal-providing substances will be captured by the immobilized binding molecules and form rings with an area proportional to the analyte concentration, and the signal-providing substances that are not bound to analyte molecules will migrate out to the periphery of the porous fluid-receiving material in the said fluid-receiving device.

In a so-called competitive embodiment of the present invention analogues of or derivatives of or fragments of or whole analyte molecules are bound to the signal-providing substances, and these will bind with the specific binding molecules in the said fluid-receiving device. In this competitive embodiment one can make use of the thermodynamic principle that at the same temperature the kinetic movement of small molecules is much faster than for large molecules. Analyte molecules from the test sample which are not bound to signal-providing substances will be able to react very quickly with the specific binding molecules, while especially particulate signal-providing substances with attached analyte molecules will react much slower than free analyte molecules. Particulate signal-providing substances will therefore in this embodiment form outer rings, while the analyte molecules from the test sample will form an inner signal free ring, preferably an inner ring with an area proportional to the analyte molecule concentration in the test.

In another embodiment of the present invention immobilized analogues of or derivatives of or fragments of or whole antigens are used in the porous fluid absorbing material in the said fluid-receiving device. Antigens are molecules that react with specific antibodies with affinity for such antigens. In this embodiment specific antibodies present in the test sample can be measured. Such specific antibodies have affinities for specific antigens. This is medically indicated especially in the case of so-called autoimmune diseases, characterized by patients producing antibodies against antigens that exist in their own body, and in the case of infectious diseases in which the patient produces antibodies against the infectious agents. Then typically competing antibodies bound to the signal-providing substances are used, these last antibodies typically produced polyclonally or monoclonally in animals or in cell cultures; or using recombinant technique in cell cultures, including bacteria cultures; or in plants. Also in this case fragments or analogues or derivatives of antibodies, or competing molecules manufactured at combinatorial chemical or biochemical libraries, including phage display may be used. A further description of these techniques and disease situations can be found in the publicly available medical and biochemical specialized literature.

Numerous different antibodies have been used in different embodiments of the present invention. Generally polyclonal antibodies are not preferred because they have the ability to aggregate the microspheres in presence of antigens, which does not happen easily when monoclonal antibodies are used, which most often binds to only one determinant of the antigens.

If the analyte is not a monomer, but has several sub-nits with the same antigen determinant, it will be an advantage to combine the coated microsphere with a reagent splitting the analyte into monomers. E.g. C-reactive protein may be split into monomers using chelating agents like DTPA and EDTA which binds the Calcium ions, and then the different sub-nits are disconnected. The concentration of the chelating agents must be higher than the concentration of calcium and manganese ions is in the blood sample to be analysed.

Furthermore, if the antigen has more than one replicate of the same epitope, particle aggregation may be induced, an another antibody reactive to an epitope which is not present at multiple locations of the antigen should be chosen.

Especially preferred are the use of pairs of monoclonal antibodies that have been tested to bind at different epitopes of the antigen without interfering with the other antibody's, binding to the antigen, and without causing aggregation of the particles.

To adapt the size of the patterns that are formed, it might be of interest to add calibrating competing substances to the said reagent. It can e.g. be of interest to add to the reagent known quantities of specific binding molecules without attached signal-providing substances, which will compete for the binding to the analyte molecule in the test sample. It can also be of interest to add known quantities of analogues of or derivatives of or fragments of or whole analyte molecules that will react with specific binding molecules which are present and thus influence the patterns that are formed.

The method relating to the present invention is based on transportation of aqueous solutions through the fluid-transmitting device and to and within the fluid-receiving device. The invention is not limited to one type of transporting force, but the capillary forces that appear when aqueous solutions come into contact with porous materials will offer a transporting force that is especially favorable for application of the method that is characteristic of the present invention. In principle gas overpressure forces or gas underpressure forces (vacuum) can also be used, often in connection with suction or pressure pumps, but this is as a rule less appropriate from a practical point of view.

To serve as fluid-transmitting materials in the said fluid-transmitting device use is typically made of the type of materials that are used in India ink pen nibs or so-called felt tip pens; typically—but not limited to—felt, sponge (natural and synthetic), but especially preferred polyethylene fibers (dense or hollow fibers), polyester fibers (dense or hollow fibers) or other plastic polymer fibers (dense or hollow fibers). When use is made of hollow fibers especially favorable capillary effects are obtained, but also dense fibers can be used since in these design varieties capillary slits are obtained between the fibers. Some materials are glued together, others are melted together or pressed together or extruded or cast or spun together.

To serve as porous or fluid-receiving materials use can be made of both hydrophilic materials and hydrophobic materials. Hydrophilic materials often offer good suction features, while hydrophobic materials often offer better features for immobilizing specific binding molecules.

Less preferred design varieties than the liquid leak proof embodiment, are design varieties making use of a more open container for mixing the reagent and the test sample. In this embodiment as well a fluid transmission device is used, but since this embodiment is not leak proof, the transmission of fluids will as a rule flow against gravity, i.e. upwards from the said container. Most typical in this embodiment the use is made of a transmission device which contains a porous fluid absorbing material which draws up fluid as a result of the capillary forces that appear when fluid comes into contact with this material, and which is not in permanent physical contact with the porous fluid-receiving material in the fluid-receiving device. This transmission device is brought into contact with both the mixture of reagent and test sample in the said container, and with the porous fluid receiving material in the fluid-receiving device, typically in the center of this fluid-receiving device so that the fluid mixture migrates radially out into the porous fluid-receiving material.

The present invention is thus provided partly by using a separate fluid transmission device as described above, which is not in fixed or permanent contact with the porous fluid-receiving material where the formed pattern is read, and partly by using a liquid leak proof container for the mixture of reagent and test sample aliquot. In this way a controlled fluid transmission is obtained, as previously used in pens and writing instruments in order to achieve controlled writing or drawing, but in the present invention in order to form controlled quantitative pattern areas for chemical quantification of analyte molecules. Preferably a combination of these two elements is used in the present invention.

In a less preferred embodiment the method relevant to the present invention is characterized by the fact that a leak proof container is used for mixing reagent and test sample, in combination with that the said fluid transmission device and the said fluid-receiving device constitute one continuous device with a continuous porous fluid absorbing material.

The said signal-providing substances preferably consist of particulate material, typically of metal colloids or of polymeric nature, alternatively of latex type, or of carbon particles, such as e.g. carbon black particles (M. Lönneberg and J. Carlsson. J. Immun. Meth., 246: (2000), 25-36.) Such colored particles are very well known in the literature and by the common specialist, and are publicly available from providers such as British Biocell, UK, and Bangs Laboratories, Indiana, USA. These firms also deliver such particles physically or chemically coated with antigens or antibodies or other binding molecules or derivatives of these or analogues of or derivatives of or fragments of or whole analyte molecules. Polymeric particles are delivered in all sizes and colors, also as fluorescent particles. The particles' size and color intensity must be adapted tothe sensitivity and capacity needed for the measurement method, as well as to the pore size of the porous fluid-receiving material in the said fluid-receiving device. Further they must be adapted to the instrument that is going to read the result or for visual direct reading, if this is to be used.

Small particles react faster than big particles and have the capacity to bind more binding molecules per mass unit of particles, while bigger particles provide stronger color or fluorescence in relation to the quantity of binding molecules that is used.

The signal-providing substances can also consist of fluorescent dyes directly conjugated to the binding molecules, but will ordinarily require a fluorescence scanner to be read. Dyes directly conjugated to the binding molecules can be used for analytes with a high concentration, and making use of the hemoglobin molecules from blood samples themselves is a special embodiment of the present invention, in such cases often with chimeric antibodies with affinity both to hemoglobin and to analyte molecules. Further enzymes can be used an signal-providing substances, but the said fluid-receiving device must in that case usually be supplied with an enzyme substrate containing additional solution, e.g. with a substrate that precipitates as a colorant.

The said binding molecules can include monoclonal or polyclonal antibodies or antigen binding fragments or derivatives of these, alternatively FAB or FAB2 or FAB′2 fragments, or polymers manufactured by way of combinatory techniques; synthetic or biological, including phage display, such as e.g. peptide binders or nucleic acid aptameres or molecules with a natural specific binding activity such as e.g. haptoglobin or intrinsic factor or folate binding proteins. If specific binding molecules are used both in the fluid-receiving device and bound to the signal-providing substances, it must be made certain that the analyte molecules can bind the specific binding molecules simultaneously.

The said reagent that is characteristic of the present invention can further with advantage contain chemicals that dissolve cells in the test sample, such as detergents and/or buffer substances that regulate pH and ionic strength or keep particles—if any—dispersed.

A further characteristic of the invention can be that the said fluid-transmitting material in the fluid-transmitting device has a pore size that holds back cells such as red or white blood cells but has a pore size that is sufficiently big to let through the said signal—providing substances.

The biological test sample to be analyzed in the present method may comprise blood, sputum, mucous, faeces, expectorates and tissues.

If a strengthening of the bindings between the signal-providing substances and the analyte molecules or between the analyte molecules and the specific binding substances in the fluid-receiving device is desirable, several types of binding molecules can be used simultaneously, alternatively with specificity for different parts of the analyte molecules.

In certain design varieties of the present invention it can be advantageous to carry out dilution or hemolysis or extraction or denaturing or separation of the test sample before it is taken into the said container. Typically substances that are present in very high concentrations may require dilution in order not to overload the binding capacity of the method related to the present invention. Other analytes, such as e.g. folates or vitamin B12, require denaturing such as boiling in order to expose the analyte molecular structure. Carbohydrate-low transferrins often have to be separated from the other isotransferrins before the quantification of the carbohydrate-low transferring can take place, and water samples must typically be concentrated or be filter extracted prior to analysis.

The reagent in the said container for mixing of reagent and test sample can in less preferable design varieties of the present invention be divided into two constituent parts, most frequently due to lacking shelf life if the reagent is combined in a solution. This can e.g. be set up by letting the two constituent parts combine immediately before use, e.g. by—but not limited to—placing one part of the reagent in a glass vial inside the container, and further that the said container is made of soft plastic and that the said glass vial is broken by compressing the said soft plastic container and that the reagent thereby is mixed. This last action can be performed prior to or after mixing in the test sample.

Alternatively the two parts of a divided reagent can be kept in two compartments that are joined together or screwed together immediately before use, or by one of the partial reagents or both the partial reagents being filled directly before use, possibly the same way as ink is filled or pumped into a fountain pen. Also if the reagent is provided as a ready-made reagent it can be filled into the container or be brought into the container in the form of a cartridge, the way ink can be brought into pens using cartridges. Another variety is to make use of refillable cartridges, which are thereafter placed into the pen-like device, or industrially manufactured cartridges containing reagents can be used.

The porous fluid-receiving material, which constitutes the whole or parts of the fluid-receiving device, can consist of different materials. Typically the material will consist of nitrocellulose with relatively large pore size, especially if the signal providing substances consist of particulate material. Further developed materials have been provided in recent years, such as the material Predator from Pall Gelman and hydrophilic and hydrophobic materials and derivatives of nylon, cellulose and other natural and synthetic polymers. Such materials are commonly available from Pall Gelman in the UK, Millipore in the US, Schleier & Schull in Germany and numerous other firms. Specific binding molecules can, however, also be immobilized on particles, often of a hydrophobic character, which disperse in the porous material, and due to their size are immobilized, i.e. are not pulled along by liquid flow, in the porous material.

The method in accordance with the present invention is further characterized by the fact that reading can take place visually or instrumentally by way of imaging, scanning or measurement of the patterns or areas of patterns and/or areas of pattern elements that appear in the said fluid-receiving device using analog or digital instruments based on visible or ultraviolet or infrared or near-infrared light, either by absorption measurement or reflection measurement or fluorescence measurement, and that the concentration of the analyte or the analytes in the test sample is determined based on these measurements. Be it that the reading takes place instrumentally or visually, the reading can be assisted by calibration indicators being imprinted on the fluid-receiving device, alternatively on overlying transparent material.

The method in accordance with the present invention is among other things suitable for analysis of concentrations of i.a.

autoantibodies such as anticadiolipin antibodies,

antibodies against antigens related to arthritis,

antibodies against HIV, rubella and other viruses as well as toxoplasmosis,

saprophytes, bacteria and other infectious agents,

hemoglobin,

albumin,

CRP,

U-albumin,

glycated albumin,

glycated hemoglobin,

ferritin,

ASAT,

ALAT,

LDH,

myoglobin,

Troponin I,

Fatty Acid Binding Protein,

amylase,

HCG,

U-HCG,

plus a long list of medical substances, such astheophyllin and different antibiotics, as well as a long list of other analytes.

The present invention further provides devices and reagents that are required for the application of the method, as well as a kit for performing the method according to the present invention. The devices related to the present invention include:

a reagent for application of the method related to the present invention,

a device for bringing the test sample, readily an aliquot of a test sample, into contact with the reagent,

a container for mixing the test sample and the reagent, preferably a liquid leak proof container, if desirable partially formed using the above mentioned device in order to bring the test sample into contact with the reagent,

a fluid-transmitting device containing a fluid-transmitting material of porous or non-porous material, preferably for liquid leak proof contacting with the said mixture of reagent and test sample in the said container,

a fluid-receiving device including a porous fluid-receiving material, wherein said porous fluid-receiving material in the said other fluid-receiving device including reagents that have specific binding affinity for the said analyte or analytes or that the said reagents consist of immobilized analyte molecules or analogues or derivatives or fragments of analyte molecules; the said reagents preferably in immobilized form, and, if desired, separate additional reagents for pretreatment or separation of test sample or admixing into the fluid-receiving device for clarification of the signal.

Best Mode

Preferred modes by which the method according to the present invention is performed are described in examples 12, 13, 14 and 17.

The following examples are presented to illustrate preferred embodiments of the present invention and shall not in any way restrict the invention.

### EXAMPLE 1

### Blue Latex Particles Coated With Monoclonal Anti-Human Myoglobin Antibodies

Wash and dialyse 60 mg Estapor blue carboxylated microspheres PSI 90-21 Batch 766, diameter 0,117 μm ; +/−0.017 μm ; COOH=164 μeq /gram against water and suspend in 2 ml water. Dialyse 5 mg monoclonal anti human myoglobin antibodies clone 7005 from Medix Biochemica Oy, Finland, in 3 ml 10 mM phosphate 15 mM NaCl buffer pH=7.2. Mix the microsphere suspendsion with 10 ml of a 10 mM phosphate 15 mM sodium chloride buffer pH=7.2. Dissolve 2 mg 1-ethyl-3(3dimethylaminopropyl) carbodiimide, supplied by Sigma Corp., U.S. in 2 ml chilled 0.25 ml of 10 mM phosphate 15 mM NaCl buffer pH=6.0. Under vigorous mixing, mix 300 μl of the 1-ethyl-3(3-dimethylaminopropyl) carbodiimide solution with the above described buffered microsphere suspension. Immediately thereafter, under vigorous mixing, admix the 5 ml solution of 5 mg monoclonal antibodies.

Keep the suspension agitating over night, and then admix 5 ml of a 0. 02 M glycin 0.01 M phosphate 0.3 M NaCl 0.1% Tween 20 (supplied from Sigma) buffer with 0.5% normal mouse serum under agitation. Wash the microspheres three times by 20 minutes centrifugation at 40000 g in a 0.05 M glycin 0.01 M phosphate 0.3 M NaCl 0.1% Tween 20 (supplied from Sigma) buffer with 0.5% normal mouse serum, and re-suspend in 0.02 M glycin 0.01 M phosphate 0.3 M NaCl 0.1% Tween 20 (supplied from Sigma) buffer with 0.5% normal mouse serum to a 2 w/v % microsphere concentration wanted. Use slight sonication to disperse the microspheres.

The concentrations of the different reagents may need some adjustments dependant on (1) the extent of carboxylation of the microspheres, (2) the concentration of antibodies wanted on the surface of the microspheres, and (3) the scale of the conjugation.

Larger volumes often need higher concentration of the 1-ethyl-3(3-dimethylaminopropyl) carbodiimide, and Merck in their Technical note<<B4 coupling on NH2 or COOH particles>>(Estapor Particle technical note 2000), in fact recommend a higher concentration of the 1-ethyl-3(3-dimethylaminopropyl) carbodiimide, which according to the inventor's experience leads to some over-conjugation and dimerisation of the microspheres. However, such over-conjugation may be compensated by having the microspheres suspended in a bigger volume, which in turn leads to higher consumption of the 1-ethyl-3(3-dimethylaminopropyl) carbodiimide. Other companies supplying coloured functionalised microspheres, like Bangs Laboratories Inc., U.S., have their own protocols that can be used for this purpose.

Dependant on the concentration of the analyte to be measured, smaller particles with high binding capacity per weight unit may be preferred, or larger microspheres with less binding capacity may be preferred in other circumstances. However, the size must be significantly lower than the pore size of the fluid receiving device to obtain free migration in the fluid receiving device.

The effectiveness of the coating with antibodies is dependant of the pI of the antibody in use. A good rule of the thumb is to use a pH of the buffer 0.5 to 0.8 pH units higher then the pI of the monoclonal antibody in use, but this is not an absolute limitation.

For different embodiments of this invention, different amounts of antibodies on the surface of the microspheres are needed. To some extent this can be done by regulating the concentration of antibodies and microspheres during the conjugation. Furthermore, the monoclonal antibodies can be diluted with nonspecific antibodies or even other proteins during the conjugations. E.g. egg albumin or bovine gamma globulins may be used for such dilution. However, attention should be payed to the fact that too much gammaglobulin or antibodies of the surface make the microspheres sticky and they may not migrate efficiently in the fluid receiving device (see below).

Before a lot of antibody-conjugated microspheres is taken into use, check that the microspheres migrate freely in the fluid receiving device, and bind to antigens immobilised on the fluid receiving device.

### EXAMPLE 2

### Blue Latex Coated With Theophylline Analogue Antigen

Make a conjugate between 8-(3-carboxypropyl)-1,3-dimethylxanthine and bovine serum albumin according to C. E. Cook & al. in Research Communications in Chemical Pathology and Pharmacology, Vol. 13, page 497-505, 1976. A lenient conjugation should be employed. The extent of conjugation can be regulated by regulating -the reactants including the 1-ethyl-3(3-dimethylaminopropyl)carbodiimide and the 8-(3-carboxypropyl)-1,3-dimethylxanthine concentration and monitored by spectroscopy methods well known in the prior art. By so doing, a degree of conjugation of 3 moles 8-(3-carboxypropyl)-1,3-dimethylxanthine per mol of bovine serum albumin was obtained. Alternativly, the product Theohpylline -8-bovine serum albumin supplied from Immune System Limited, UK may be used, although less optimal.

Conjugate Estapor blue carboxylated microspheres PSI 90-21 to anti-bovine albumin monoclonal antibody supplied by Chemicon Inc., California, using the method described in example 1. Dissolve the conjugate between 8-(3-carboxypropyl)-1,3-dimethylxanthine and bovine serum albumin in the assay solution described in example 4, at a concentration of 0.1 mg/ml of the conjugate and 2 mg particles per ml. Leave the suspension to stand for 10 minutes, thereafter washed three times in assay solution with 0.25% v/v mouse normal serum by centrifugation at 30.000 g, and suspend thereafter in assay buffer by gentle sonication.

An even better alternative is the use of a direct binding of the 8-(3-carboxypropyl)1,3-dimethylxanthine bovine serum albumin conjugate to the blue latex using the coupling method described in example 1. However, to assure that not all amine groups are blocked in the bovine serum albumin and that the isoelectric point of the bovine serum albumin is not lowered too much, thus preventing a poor coupling efficiency, a low degree of conjugation of 8-(3-carboxypropyl)-1,3-dimethylxanthine to the bovine serum albumin on beforehand is necessary.

Before a lot of protein coated microspheres is taken into use, one ought to check that the microspheres migrate freely in the fluid receiving device, and bind to antigens immobilised on the porous material in the fluid receiving device, see example 8.

### EXAMPLE 3

### Blue Latex Coated With Streptavidin And Biotinylated With Monoclonal or Polyclonal Antibodies

Wash and dialyse a suspension of Estapor blue carboxylated latex microspheres from Merck Eurolab, Product number K1010, mean diameter 185 nm, to contain 10 mM phosphate buffer pH=6.0 with 15 mM NaCl and 3.5 vol. % particles. Dialyse 5 mg streptavidin (Sigma) against the same buffer solution.

Dissolve 2 mg 1-ethyl-3(3-dimethylaminopropyl) carbodiimide in chilled 0.25 ml of said 10 mM phosphate buffer pH=6.0 with 15 mM NaCl, and add immediately 40 μl to 1.5 nil of said particles suspension under mixing, and thereafter 6 ml of said buffer solution and in addition comprising 2 mg streptavidin is admixed. Agitate the suspension at room temperature for 2 hours, and then keep the suspension agitating over night. Thereafter, admix 5 ml of a 0.02 M glycin, 0.01 M phosphate, 0.3 M NaCl, 0.1% Tween 20 (supplied from Sigma) buffer with 0.5% normal mouse serum under agitation.

Wash the microspheres three times by 20 minutes centrifugation at 40.000 g in 0.02 M glycin 0.01 M phosphate, 0.3 M NaCl, 0.1% Tween 20 (supplied from Sigma) buffer with 0.5% normal mouse serum, and re-suspend the microspheres in 0.02 M glycin 0.01 M phosphate 0.3 M NaCl 0.1% Tween 20 (supplied from Sigma) buffer with 0.5% normal mouse serum to the microsphere concentration wanted. Use slight sonication to disperse the microspheres.

The concentrations of the different reagents may need some adjustments dependant on (1) the extent of carboxylation of the microspheres, (2) the concentration of antibodies wanted on the surface of the microspheres, and (3) the scale of the conjugation.

Larger volumes often need higher concentration of the 1-ethyl-3(3-dimethylaminopropyl) carbodiimide. In fact, Merck in their Technical note <<B4 coupling on NH2 or COOH particles>>(Estapor Particle technical note 2000), recommend a higher concentration of the 1-ethyl-3(3-dimethylaminopropyl) carbodiimide. According to the inventor's experience, this may lead to some over-conjugation and dimerisation of the microspheres, which, however, may be compensated by having the microspheres suspended in a bigger volume. This may in turn lead to higher consumption of the 1-ethyl-3(3-dimethylaminopropyl) carbodiimide. Other companies supplying coloured functionalised microspheres, like Bangs Laboratories Inc., U.S., have their own protocols that may be used for such purposes. Dependant of the concentration of the analyte to be measured, smaller particles with high binding capacity per weight unit may be preferred, or larger microspheres with less binding capacity may be preferred in other circumstances. However, the size must be significantly lower than the pore size of the fluid receiving device to obtain free migration in the fluid receiving device.

Since avidin is less hydrophilic than avidin, avidin is often preferred over streptavidin. The conjugation is very much the same; however, a higher pH is chosen because avidin has a much higher pI. A 0.1 M borate buffer pH=9.0 can then be used as a coupling buffer, however the 1-ethyl-3(3-dimethylaminopropyl) carbodiimide will then be hydrolysed very rapidly, and some better cooling and higher concentration will often be necessary.

Further, to biotinylate the antibody wanted, dialyse 5 mg monoclonal antibody in 1 ml solution against 0.15 M sodium chloride 0.1 M phosphate buffer pH =7.2.

Dissolve 2 mg sulfosuccinimidyl-6-(biotinamido)hexanoate from Pierce Chemical Company in 10 ml of cold distilled water, 2-8° C., and add 100 μl of the resulting sulfo-NHS-biotin solution to the 1 ml antibody solution, while Vortex-mixing. Place the tube in a refrigerator for 2 hours. Separate the antibodies with biotin coupled from free biotin by size exclusion chromatography on a 30 cm Superose 6 column (Amersham Pharmacia Biotech, UK) using a 0.1 M phosphate 0.15 M sodium chloride buffer at pH=7.2 as eluant. Collect the protein fraction eluted in front of the free biolin fraction, using a UV monitoration unit. Altrnatively, dialyse against the same buffer solution using a dialysing membrane with a exclusion size of 7000 Dalton, e.g. the cassette Slide-A-lyzer from Pierce Chemical Company.

The biotin incorporation in the antibody can be monitored by the use of the method taught by N. M. Green in Biochem J. 94, 23c-24c, 1965. The above described procedure yielded 0.2 moles of biotin per mole of antibody.

The antibody in use can be of mouse, sheep, hen egg, sheep, goat, human origin or from another species, and can be of monoclonal or polyclonal origin. Mostly monoclonal antibodies are preferred, since polyclonal antibodies have a tendency to aggregate when antigens are present, but if concentrations are adjusted, also polyclonal antibodies can be used. Also immuno-active fragments of antibodies can be used, so also peptides and aptamers and other binding substances with the wanted binding specificity, but the biotinylation chemistry must then be modified. The number of biotin moieties per molecules of antibody must be substantially lower than 1 in average, since more than 1 biotin moiety in an antibody molecule will aggregate the microspheres even without presence of antigens. Furthermore, if lower fraction of active antibodies are wanted, the specific antibodies can be diluted by non-specific antibodies or even other proteins prior to the biotinylation (however, then calculation of degree of biotinylation becomes more difficult).

Mix the biotinylated antibodies to the avidin or streptavidin coated microspheres to obtain microspheres with specific antibodies attached. The amount of antibodies bound to the microspheres can be adjusted dependent on how much biotinylated antibodies added. By adding an excess of biotinylated antibody, it is possible to measure (after separation by centrifugation) the free biotinylated antibodies in the solution not bound to the microspheres, using the method taught by N. M. Green in Biochem J. 94, 23c-24c, 1965.

Then wash the microspheres three times by 20 minutes centrifugation at 40000 g in 0.02 M glycin 0.01 Mphosphate 0.3 M NaCl 0.1% Tween 20 (supplied from Sigma) buffer with 0.5% normal mouse serum, and re-suspend in 0.02 M glycin 0.01 M phosphate 0.3 M NaCl 0.1% Tween 20 (supplied from Sigma) buffer with 0.5% normal mouse serum to the microsphere concentration wanted. Use slight sonication to disperse the microspheres.

The streptavidin or avidin coating is less favourable than direct antibody binding to the microspheres, since presence of some double-biotinylated antibodies may cause aggregation of particles.

Before a lot of microspheres are taken into use, one ought to check that the microspheres migrate freely in the fluid receiving device, and bind to antigens immobilised on the fluid receiving device.

### EXAMPLE 4

### Gold Colloid Coated With Anti-Human Albumin Antibodies

Mix for 20 minutes 10 ml of 1% gold chloride in distilled water with 1 l of boiling distilled water, 10 ml of 34 mM sodium citrate, pH adjusted to pH=4.2.Colloidal gold is formed. Allow the suspension to cool to room temperature. Ad and mix 1 ml of 1% PEG 20.000, and adjust pH to pH=7.2. The size of the gold colloid particles is measured using a conventional technique, e.g. by measurement of the ratio of optical density of 540 nm and 600 nm. The method may be adjusted to obtain mean particle size between 30 and 50 nm. Glassware to be used must be siliconized. Label the gold colloid particles with monoclonal anti human albumin antibodies clone 6501 from Medix Biochemical OY, Finland, using the method described by Slot and Geuze in Eur. J. Cell. Biol. 38: 87-93, 1985, to the saturation point according the same method. Then, typically, but not limited to, the labelled gold colloids are suspended at a protein concentration of 10 ug/ml in a 10 mM HEPES buffer solution at pH=7.4, comprising 0.3 M mannitol, 0.05% PEG 20000.

Other antibodies can be used in the place of the said anti-albumin antibodies, but slight alterations of the procedure may be necessary.

Before a preparation of coated colloid particles is taken into use, one ought to check that the colloids migrate freely in the fluid receiving device, and bind to antigens immobilised on the fluid receiving device

### EXAMPLE 5

### Florescent Cyanin-5-Theophyllin Conjugate

Make a synthesis of 8-(3-carboxypropyl)-1,3-dimethylxanthin anhydride as described in Research Communications in Chemical Pathology and Pharmacology, vol. 13, p. 497-505, 1976, and in Clinical Chemistry. vol. 27, page 22-226, 1981. Dissolve diaminopropanol in water-free tetrahydrofuran. In another flask, dissolve half of the equiM amount of the said 8-(3-carboxypropyl)-1,3-dimethylxanthin anhydride in water-free tetrahydrofuran. Add the said 8-(3-carboxypropyl)-1,3-dimethylxanthin anhydride solution drop-wise to the diaminopropanol solution while stirring, and let the resulting solution react over night at room temperature. Optionally purify the resulting adduct by HPLC chromatography using conventional techniques well known to the skilled man of the art, if less consumption of activated cyanine dye is wanted (see below).

Thereafter, dissolve 6 times the M amount which was used for diaminoprcpanol, of Cy5 Fluorolink activated cyanin dye supplied from Amersham Pharmacia Biotech, U.K., in water-free tetrahydrofuran, and add it to the previously described solution while stirring. Leave the resulting mixture to react over night a room temperature in darkness. In this way, a stock solution of non-pure 8-(3-carboxypropyl)-1,3-dimethylxanthin adduct with Cy5 Fluorolink activated cyanin dye with a water-soluble diaminopropanol spacer is obtained. Purify the resulting 8-(3carboxypropyl)-1,3-dimethylxanthin adduct with Cy5 Fluorolink activated cyanin dye with a water-soluble diaminopropanol spacer by means of thin layer chromatography in silica gel using n-butanol: acetic acid:water in a 1:1:1 mixture, however ad just the volumes of n-butanol, acetic acid and water in the elution mixture depending on the quality of the silica gel plates to obtain good separation. After elution by conventional technique, dry the silica gel plate and inspect visually and by UV lamp (and optionally using ninhydrin spray in parallel experiments) to identify the spot of 8-(3-carboxypropyl)-1,3-dimethylxanthin adduct with Cy5 Fluorolink spot. Isolate the silica gel 8-(3-carboxypropyl)-1,3-dimethylxanthin adduct with Cy5 Fluorolink by scissors or spatula. Suspend the isolated silica gel in 50% v/v acetic acid whereby 8-(3-carboxypropyl)-1,3-dimethylxanthin adduct with Cy5 Fluorolink is eluted into the solution. The silica gel settles in the botton of the tube. Decant off the acetic acid solution with the purified 8-(3-carboxypropyl)-1,3-dimethylxanthin adduct with Cy5 Fluorolink, and remove the acetic acid by evaporation under low atmospheric pressure. Alternatively, and for up-scaling, conventional HPLC separation techniques well known to the skilled man of the art may be used in stead of thin layer chromatography.

### EXAMPLE 6

### Assay Solution

An example of an assay buffer is to make a 0.1 M aqueous phosphate buffer, add sodium chloride to 0.3 M concentration, furthermore add the detergent Triton X-100 (Supplied from Sigma , U.S.) to a final concentration of 0.1%, and adjust the pH to pH=7.4 using hydrochloric acid or sodium hydroxide in conventional manner. Signal forming particles according to any of the examples 1 to 3 is then added, typically but not limited to from 0.01 to 1.0% v/w of latex particles, or colloidal gold at a concentration of 1-25 ug immunoglobulin labelled to the colloid per ml solution. 0.25% of normal serum from one of the species from which the antibodies in use are derived, are added to the solution. 0.1% w/v bovine gammaglobulin can be used in stead of mouse serum, unless it interferes with the assay, as described in example 8.

### EXAMPLE 7

### Container for Signal-Providing Substances and a Liquid Transmitting Device Containing a Liquid-Transmitting Material to be Introduced into the said Container

As illustrated in FIG. 2 one embodiment of the said device may comprise a stopper 6 with a built-in capillary 7, such as e.g. a capillary holding 5 μl fluid, a sealing sleeve 8, a liquid proof container 9, a ball 10 sealing the port in the bottom of the container 9 wherein the ball is housed in a valve seat 11 which is formed to receive a wick or felt tip guide 12 in a sealing connection. The wick or felt tip 13 in a sealing and sliding connection in the wick or felt tip guide 12, and the tip is protected by a cap 14.All parts of the device is produced by suitable materials such as e.g. plastic, except the wick or felt tip which is made of a fluid-transmitting material. The container 9 is filled with a reagent 15 as illustrated in FIG. 3. The sample of a biological fluid, such as e.g. blood is filled in the capillary 7, heparinized or not, which in this embodiment holds 5 μl, but which may be constructed to hold other volumes, the stopper 6 pressed down into the container 9, and the test sample and reagent 15 are mixed by moving the container 9. The volume of the test sample is adapted to the volume of the reagent in the container 9. The present invention further comprises an embodiment wherein the test sample is filled in a capillary, heparinized or not, which thereafter is placed in the container 9. After closing the container with the stopper 6 the test sample is mixed with the reagent by moving the container adequately, and the contact with the test sample reagent mixture and the fluid receiving material 17 through the fluid-receiving device is provided either by;

simultaneously bringing the fluid-transmitting material in contact with the said mixture and the fluid-receiving material,

bringing the fluid transmitting material first in contact with the said mixture, then in contact with the fluid receiving material,

bringing the fluid-transmitting material first in contact with the fluid-receiving material, then in contact with the said mixture.

### EXAMPLE 8

### HiFlow Nitro-Cellulose Filter Material Coated with Anti-Theophylline Antibodies

Isolate the IgG fraction of sheep anti-theophylline serum from Immune System Limited, U.K., by conventional techniques well known to the skilled man of the art, e.g. by aminonium sulphate precipitation or by the use of a Protein A column from Amersham Pharmacia Biotech. Thereafter dialyse the antibodies in 10 mM phosphate 15 mM NaCl, buffer pH=7.2, and thereafter dissolve the antibodies in a 10 mM ammonium acetate solution with 2.5% v/v ethanol. If low binding capacity is wanted, other proteins like albumin or casein can be added in addition, which will compete with the specific antibodies in the subsequent adsorption process.

The said solution is either sprayed on the Hi-Flow material or the sheets are soaked in the said solution. Thereafter, dry the sheets at 37° C. for two hours. Further, wash the sheets by soaking and agitation at room temperature in a 10 mM ammonium acetate solution with 2.5% v/v ethanol with 0.01% w/v of 3-3(-cholamidopropyl)dimethylamonio-2-hydroxo-1-propane sufonate (from Pierce Chemical Company, U.S.)

The concentration of the specific antibody varies according to the needed binding capacity in the said porous material. To determine the concentration necessary in this example, a serum sample 10 μl comprising 50 ng of theophylline is mixed with 2 ml of the assay solution of example 4 comprising a 0.1% suspension (2 mg microspheres) of the blue latex microspheres described in example 2.

The appropriate concentration of the antibodies in the Hi-Flow Plus HF12004 is determined as follows: Allow the said mixture to migrate into the porous material to be used in the a fluid receiving device. The theophylline present in solution reacts much faster with the antibodies immobilised in the porous material than the conjugates on the microspheres, and blocks the binding of the blue micrcspheres in the filter material. High concentration of specific antibodies immobilised on the porous material lead to rapid binding of free theophylline in solution in smaller areas, while low concentrations lead to larger areas are needed for binding of the faster reacting free theophylline in the solution areas; with reference to FIG. 4, the area (18) is smaller when high concentration of antibody is immobilised, and larger when low concentration of antibody is immobilised. When the assay solution has been depleted from free theophylline in solution in the migration process, the blue microspheres, with 8-(3-carboxypropyl)-1,3-dimethylxanthine conjugate with bovine serum albumin bound to the blue microspheres, start to react with the immobilised monoclonal anti-theophylline antibodies which have not been blocked with free theophylline; with reference to FIG. 4 in area (17) outside (18). In other words, a darker blue ring (17) is produced outside the inner whiter area (18). Adjust the concentration of specific antibodies to obtain a binding capacity of free theophylline in the porous HiFlow material size of the area wanted for a specific concentration of the analyte. In this example it was found appropriate to immobilise 25-50 μg antibody per square cm of material. (The binding capacity for the HiFlow Plus HF12004 after immobilisation of antibodies can be determined by conventional methods found in the literature, e.g. using radio-labelled antigens or enzyme-linked antigens in combination with competing known standard solutions of antigen). If very high binding capacity is wanted, the use of monoclonal antibodies from Immun System Ltd., U.K., is recommended.

Alternatively, other porous materials may be used, e.g. the Predator PREDL3R filter material from Pall Gelman, U.K., that are able to bind binding proteins at a high concentration and chemical activity, and with a pore size that allows a free migration of the signal carrying antibody conjugates, e.g. coloured latex particles or gold colloid particles.

Most such porous materials need some wetting agents to perform well, but the detergents like CHAPSO may often be omitted, or other detergents may be used, although in low concentrations. Also, the effect on the antibodies ability to bind must be checked, using techniques well-known to the person skilled in the art.

### EXAMPLE 9

### Porous Material to be used in a Fluid Receiving Device for Quantitation of Myoglobin

Slit Hi-Flow Plus HF12004 supplied from Millipore into sheets of suitable size.

Dissolve monoclonal mouse anti-myoglobin antibodies clone 7004 supplied from Medix Biochemica OY, Finland, in a 10 mM ammonium acetate solution with 2.5% vol./vol. ethanol. If low binding capacity is wanted, other proteins like albumin or casein can be added in addition, which will compete with the antibodies in the subsequent adsorption process. Said solution is either sprayed on the Hi-Flow material or the sheets are soaked in the said solution. Then, dry the sheets at 37° C. for two hours. The sheets are thereafter washed by soaking and agitation at room temperature in 10 mM ammonium acetate solution with 2.5% vol./vol. ethanol with 0.01% w/v of 3-(3-cholamidopropyl)dimethylamonio-2-hydroxo-propane sulfonat (from Pierce Chemical Company, U.S.)

The concentration of the specific antibody varies according to the needed binding capacity in the said porous material. If low binding capacity is wanted, other proteins like albumin or casein can be added in addition, which will compete with the antibodies in the subsequent adsorption process. In this example, a normal serum sample of 10 μl comprising 10 ng of myoglobin is mixed with 2 ml of the assay solution of example 4 comprising a 0.1% suspension (2 mg microspheres) of the blue latex microspheres with monoclonal anti-myoglobin described in example 1, with a total binding capacity of myoglobin higher than 10 ng (i.e. much higher binding capacity is employed. The binding capacity for the particles in the assay reagents can be determined by conventional methods found in the literature, e.g. using radio-labelled antigens and measure the binding capacity after isolation of the particles, or by e.g. equilibrium dialysis.)

Determine the appropriate concentration of anti human myoglobin antibodies for immobilisation in the porous material as follows: Allow the said mixture to migrate into the porous material to be used in the fluid receiving device. High concentration of specific antibodies immobilised on the porous material lead to trapping of the coloured particles in small areas, while low concentrations lead to larger areas of trapping of corresponding particle solution. Adjust the concentration of specific antibodies to obtain the binding capacity leading to a size of the area wanted for a specific concentration of the analyte.

Alternatively, other porous materials may be used, e.g. the Predator PREDL3R filter material from Pall Gelman, U.K., that are able to bind binding proteins a a high concentration and chemical activity, and with a pore size that allows a free migration of the signal carrying antibody conjugates, e.g. coloured latex particles or gold colloid particles.

Most such porous materials need some wetting agents to perform well, but the detergents like CHAPSO may often be omitted, or other detergents may be used, although in low concentrations. Also, the effect on the antibodies ability to bind must be checked, using techniques well-known to the person skilled in the art.

### EXAMPLE 10

### Porous Material to be Used in a Fluid Receiving Device for Quantitation of Urine Albumin

Slit Hi-Flow Plus HF12004 supplied from Millipore into sheets of suitable size.

Immobilise monoclonal mouse anti-human albumin antibodies clone 6502 supplied from Medix Biochemica OY, Finland, on Hi-Flow Plus HF12004 using the method described in example 9.

The concentration of the specific antibody varies according to the needed binding capacity in the said porous material. If low binding capacity is wanted, other proteins like casein can be added in addition, which will compete with the antibodies in the subsequent adsorption process. In this example, a normal serum sample of 10 μl comprising 0.02 micrograms of human albumin is mixed with 2 ml of the assay solution of the gold colloid particles described in example 4, a protein concentration of 10 ug/ml in a 10 mM HEPES buffer solution at pH=7.1, comprising 0.3 M mannitol, 0.05% PEG 20000. The appropriate concentration of monoclonal anti-albumin antibodies from clone 6502 immobilised in the Hi-Flow Plus HF12004 is determined as follows: Allow the said mixture to migrate into the porous material to be used in the fluid receiving device. High concentration of specific antibodies immobilised on the porous material lead to trapping of the coloured particles in small areas, while low concentrations lead to larger areas of trapping of corresponding particle solution. Adjust the concentration of specific antibodies to obtain the binding capacity leading to a size of the area wanted for a specific concentration of the analyte.

Alternatively, other porous materials may be used, e.g. the Predator PREDL3R filter material from Pall Gelman, U.K., that are able to bind binding proteins at a high concentration and chemical activity, and with a pore size that allows a free migration of the signal carrying antibody conjugates, e.g. coloured latex particles or gold colloid particles.

Most such porous materials need some wetting agents to perform well, but the detergents like CHAPSO may often be omitted, or other detergents may be used, although in low concentrations. Also, the effect on the antibodies ability to bind must be checked, using techniques well-known for the person skilled in the art. Detergents effects on gold colloids are especially undesirable, so washing in a detergent-free corresponding buffer after applying the detergent is then performed.

### EXAMPLE 11

### A liquid Receiving Device

As illustrated in FIG. 4 one embodiment of the fluid-receiving device may comprise a circular tray 16 made of suitable materials such as e.g. plastic, wherein the fluid-receiving material 17 is located. The grey area 18 illustrates the pattern appeared as a result of the signal providing substances' dissemination in the porous material 17. In one embodiment of the present invention the tray 16 may comprise a circular chip, made of clear plastic, with a diameter of 3 cm, however other measures are within the idea of the invention. The tray is equipped with a cavity or hole in the center for placing the fluid-transmitting device. Furthermore calibration lines, circles or any other form suited to the form of the fluid-receiving tray 16, may be printed on the tray. The fluid receiving material 17, e.g. impregnated with immobilized antibodies as described above, is mounted in the tray. The pattern exhibited on the fluid receiving-material will depend on the type of analysis performed. In one embodiment the complex of the antigen in the test sample and the antibody-coloured particle in the said reagent pattern will provide a sandwich complex consisting of antigen-antibody-particle-immobilized antibody on the fluid-receiving material with the form e.g. exhibited in FIG. 4.

In another embodiment which constitutes a competitive assay, wherein the said reagent contains antigens connected to coloured particles there will be a competitive binding on the fluid-receiving material between the free antigens in the test sample and the antigen-coloured particle complex. Due to the larger molecular weight of the last complex the binding to the immobilized antibody occurs later and will exhibit a circular band outside the circular band depicting the complex of free antigens and immobilized antibody (not illustrated).

In the last embodiment, such as described in example 17, which also constitutes a competitive assay, the antigens in said reagent is connected to fluorescent moieties and the complex has a molecular weight similar to the free antigen in the test sample. In the resulting competitive binding on the fluid-receiving material there will be no size induced delay in the binding of the antigen-fluorescent particle to the immobilized antibodies and the pattern formed is similar to that formed in the said first embodiment.

### EXAMPLE 12

### Quantitation of Theophylline in Whole Blood

5 μl of blood is sucked into the Heparin-treated capillary channel at the top of reagent container according to example 11. Push down the capillary part, and the content of the capillary is by gently shaking mixed with the reagent in the reagent container comprising 1 ml of the assay solution of example 6 comprising a 0.1% suspension (1 mg microspheres) of the blue latex microspheres described in example 2 and 0.25% v/v normal mouse serum. In this step the theophylline reacts with the antibodies present in excess on the blue latex, and the red blood cells are lysed by triton, and most other particles of blood are also dissolved or dispersed by the Triton X-100.

In some embodiments of the invention the suspension is allowed to stand to react for 1 to 5 minutes (especially if the analyte to be measures is in a very low concentration). However, in most embodiments, including the present example, the binding is close to complete during the time of the shaking.

Introduce thereafter the fluid transferring device described in example 7 above into the reagent container comprising the blood sample/reagent mixture. Place the fluid transferring device's other end in the centre of the fluid receiving device described in example 11 above, with filter material with immobilised anti theophylline antibodies described in example 8 above. Allow the sample/ reagent mixture to flow through the fluid transferring device into the said fluid receiving device. The theophylline present in the solution reacts much faster with the antibodies immobilised in the porous material than the conjugates on the microspheres, and compete efficiently with the binding of the blue microspheres in the filter material. When the assay solution has been depleted from free theophylline in solution in the migration process, the blue microspheres, with 8-(3-carboxypropyl)-1,3-dimethylxanthine conjugate with bovine serum albumin bound to the blue microspheres, bind without the said competetion with the immobilised monoclonal anti-theophylline antibodies, and produces a darker blue ring outside the inner less dense area. When the porous filter material in the fluid receiving device has been saturated with liquid, the flow of liquid through the fluid transferring device stops automatically. Inspect the size of the less dense area inside the ring of the blue latex particles formed in the fluid receiving device, and compare to the indicators printed on the surface of the fluid receiving device. To establish a calibrated reliable quantitative method in the wanted concentration range of theophyllin in whole blood, the use of calibrators of human whole blood with known concentrations of human theophylline is preferred. Select the microspheres concentration in the assay solution necessary and the conditions necessary to obtain the size of the circles formed by the blue latex particles wanted to be appropriate for inspection, and to assign appropriate concentration values to the indicators printed on the surface of the said fluid receiving device.

### EXAMPLE 13

### Quantitation of Myoglobin in Human Whole Blood

10 μl of blood is sucked into the Heparin-treated capillary channel, FIG. 2,7, at the top of reagent container according to example 11. Push the capillary part down, and the content of the capillary is by gently shaking mixed with the reagent in the reagent, FIG. 3A 15, container comprising 1 ml of the assay solution of example 6 comprising a 0.1% suspension (1 mg microspheres) of the blue latex microspheres with monoclonal anti-myoglobin described in example 1 and 0.25% v/v normal mouse serum. In this step the myoglobin reacts with the antibodies present in excess on the blue latex, and the red blood cells are lysed by triton, and most other particles of blood are also dissolved or dispersed by the Triton X-100. In some embodiments of the invention the suspension is allowed to stand to react for 1 to 5 minutes (especially if the analyte to be measures is in a very low concentration). However, in most embodiments, including the present example, the binding is close to complete during the time of the shaking. Thereafter, introduce the fluid transferring device described in example 7 above as in FIG. 3D-E, into the reagent container comprising the blood sample/reagent mixture as in FIG. 3B-C. Then, place the other end of the fluid transferring device in the centre of the fluid receiving device described in example 11 above and FIG. 4, with filter material with immobilised anti human myoglobin antibodies described in example 9 above. Allow the sample/reagent mixture to flow through the fluid transferring device into the said fluid receiving device. When the porous filter material in the fluid receiving device has been saturated with liquid, the flow of liquid through the fluid transferring device stops automatically.

Inspect the size of the ring of the blue latex particles (FIG. 4, 18) formed in the fluid receiving device, and compare the size to the indicators printed on the surface of the fluid receiving device. To establish a calibrated reliable quantitative method in the wanted concentration range of human myoglobin in whole blood, the use of calibrators of human whole blood with added known concentrations of human myoglobin is preferred. Select the microsphere concentration in the assay solution necessary and the conditions necessary to obtain the size of the circles formed by the blue latex particles wanted to be appropriate for inspection, and assign appropriate concentration values to the indicators printed on the surface of the said fluid receiving device.

### EXAMPLE 14

### Quantitation of Albumin in Urine

Seal 1 ml of the anti human albumin antibody coated gold colloid particles from example 4 in 10 mM HEPES buffer solution at pH=7.4, comprising 0.3 M mannitol, 0.05% PEG 20000, into the container described in example 7 above and in FIG. 2,9.

Draw a 10 μl urine sample (or a less preferred a diluted urine sample) from a patient suffering from diabetic renal disease into the self-calibrating capillary sampling device described in example 7 above and FIG. 2, 7, and introduce said sample into the reagent container comprising said anti human albumin antibody coated gold colloid particles suspension as in FIG. 3B-C. Said container is shaken and allowed to stand for 2 minutes to let the gold colloids bind to the albumin present in the sample.

Introduce thereafter the fluid transferring device described in example 7 above into the reagent container comprising the urine sample/reagent mixture; see FIG. 3D-E. Place the fluid transferring device's other end in the centre of the filter material with immobilised anti human albumin antibodies described in example 10 above, mounted in the holder to become the fluid receiving device described in example 11 above. Allow the sample/reagent mixture to flow through the fluid transferring device into the said fluid receiving device. When the porous filter material in the fluid receiving device has been saturated with liquid, the flow of liquid through the fluid transferring device stops.

Inspect the size of the ring (FIG. 4, 18) of the gold colloid particles formed in the fluid receiving device, and compare it to the indicators printed on the surface of the fluid receiving device.

To establish a calibrated reliable quantitative method in the wanted concentration range of human albumin in urine, the appropriate binding capacity of the immobilised anti human albumin antibodies in the porous material described in example 10 above must be selected. The expected concentration of albumin in urine is very different in different patient groups, so a calibration for the intended patient group is necessary. This example functions well in concentrations varying from 0 to 200 mg per liter urine, and even concentrations up to 500 mg/ml can be measured well. If the concentration of albumin is very high, the binding capacity of the gold colloids are saturated, and a much paler area in the centre of the fluid receiving device are seen, where free albumin not bound to gold colloids bind first to the immobilised monoclonal antibodies in the fluid receiving material. By the use of calibrators of human urine with known concentrations of human albumin, the skilled person of the art can select the conditions necessary to obtain an outer diameter of the circles formed by the gold colloid particles wanted, the intensity of the colour to be appropriate for inspection, and to assign appropriate concentration values to the indicators printed on the surface of the said fluid receiving device.

If a product is intended for patients with very high albumin concentrations in urine, the size of the capillary urine sampler should be reduced, e.g. to 2 μl, and the concentration of the anti human albumin antibodies coated gold colloid particles should be increased.

### EXAMPLE 15

### Measurement of Anti-Thyroid Peroxidase Antibodies in a Sample of Whole Blood

Anti-human thyroid monoclonal antibodies are purchased from HyTest, U.K., and conjugated to carboxylated blue latex according to the method of example 1.

Human thyroid peroxidase enzymes in a proteinaceous solution is bought from The Binding Site Ltd., U.K, and coated on Hi-Flow Plus HF12004 from Millipore, according to the method of example 9. Although this material contains carrier protein and significant amounts of serum albumin, this material behaves well for coating thyroid peroxide enzymes. If higher concentrations of immobilised thyroid peroxidase in the porous material is wanted, remove the serum albumin from the product from HyTest by immunochromatogrpahy according to methods well known in the prior art.

10 μl of blood is sucked into the Heparin-treated capillary channel at the top of reagent container according to example 7. Push the capillary part down, and the content of the capillary is by gently shaking mixed with the reagent in the reagent container comprising 1 ml of the assay solution of example 6 comprising a 0.1% suspension (1 mg microspheres) of the blue latex microspheres with monoclonal anti-thyroid peroxide described above and 0.25% v/v normal mouse serum. In this step, anti-thyroid peroxide antibodies from the patients sample reacts with the antibodies present in excess on the blue latex, and the red blood cells are lysed by triton, and most other particles of blood are also dissolved or dispersed by the Triton X-100. Allow the suspension to stand to react for 3 minutes. Introduce thereafter the fluid transferring device described in example 7 above into the reagent container comprising the blood sample/reagent mixture. Then, place the other end of the fluid transferring device in the centre of the fluid receiving device described in example 11 above, with filter material with immobilised human thyroid peroxidase. The sample/reagent mixture is allowed to flow through the fluid transferring device into the said fluid receiving device. When the porous filter material in the fluid receiving device has been saturated with liquid, the flow of liquid through the fluid transferring device stops automatically.

Inspect the size of the ring of the blue latex particles formed in the fluid receiving device, and compare it to the indicators printed on the surface of the fluid receiving device. To establish a calibrated reliable quantitative method in the wanted concentration range of anti-thyroid antibodies in whole blood, the use of calibrators of human whole blood with known concentrations of anti-thyroid peroxide antibodies is preferred. Select the thyroid peroxide antigen concentration necessary in the porous fluid receiving material for the clinical group of patients to be measured, and assign the appropriate concentration values to the indicators printed on the surface of the said fluid receiving device using the calibrator of known content of anti-thyroid peroxide antibodies.

### EXAMPLE 16

### Measurement by Means of Imaging or Scanning Equipment

On the surface of the fluid receiving device described in example 11 for use of in example 12,13,14 and 15, it can be printed calibrating indicators to visually read the content of the analyte in question. To obtain both more exact quantitation and better documentation of the result of the assay, the fluid receiving device is scanned or depicted. In its simplest form, place the device on a flatbed scanner connected to a personal computer. In a more sophisticated form, depict the device by means of a digital camera or a scanner, or a fluorescence scanner. Mostly two-dimensional scanners have been used, but the use of a linear scanner is also possible to measure e.g. diameters of round spots or length of a rectangular migration path. A detailed description of such scanners, cameras and software for measurements of the fluid receiving device is given in patent application PCT/GB98/00120 by Bremnes and Sundrehagen.

### EXAMPLE 17

### Fluorescent Measurement of Theophylline Concentration in Whole Blood

5 μl of blood is sucked into the Heparin-treated capillary channel at the top of reagent container according to example 11. Push the capillary part down, and gently shake the content of the capillary to be mixed with the reagent in the reagent container comprising 1 ml of the assay solution of example 6 comprising fluorescent Cyanin-5-theophyllin conjugate described in example 5. and 0.25% v/v normal mouse serum.

Then, introduce the fluid transferring device described in example 7 above into the reagent container comprising the blood sample/reagent mixture. Then, place the fluid transferring device's other end in the centre of the fluid receiving device described in example 11 above, with filter material with immobilised anti-theophylline antibodies described in example 8 above. The sample/reagent mixture is allowed to flow through the fluid transferring device into the said fluid receiving device. The theophylline, present in solution, competes with the binding of fluorescent theophylline conjugate in the filter material. When the porous filter material in the fluid receiving device has been saturated with liquid, the flow of liquid through the fluid transferring device stops automatically. The theophylline from the blood sample and the fluorescent conjugate of theophylline is bound in the fluid receiving device (see FIG. 4, (18)) with an area proportional to the sample's concentration of theophylline. The fluid receiving device is scanned with a fluorescence scanner with excitation wavelength of 648 nm, and measure the fluorescence of the cyanine-5 on the surface of the fluid receiving device. By software computing according to Bremnes and Sundrehagen PCT/GB98/00120, the area of the fluorescence is depicted and measured. To establish a calibrated reliable quantitative method in the wanted concentration range of human transferrin in whole blood, use calibrators of human whole blood with known concentrations of human theophylline. The amount of Cyanin-5-theophylline conjugate in the reagent container must be adjusted to the sensitivity of the fluorescence scanner.

### EXAMPLE 18

### Fluorescent Measurements of Theophylline Concentration in Whole Blood Using Fluorescent Microspheres

Carboxylated microspheres prod. no. PC04N dyed with Cyanin-5 fluorescent dye is bought from Bangs Laboratories Inc., U.S., and coated wih theophylline analogue antigen according to example 2.

5 μl of blood is sucked into the heparin-treated capillary channel at the top of reagent container according to example 11. Push down the heparinized capillary part, and the content of the capillary is by gently shaking mixed with the reagent in the reagent container comprising 1 ml of the assay solution of example 6 and 0.25% normal mouse serum and comprising theophylline coated microspheres according to example 2, except that the above said Cyanin-5-dyed microspheres from Bangs Laboraotries are used in the place of the Estapor blue microspheres of example 2.

Introduce thereafter the fluid transferring device described in example 7 above into the reagent container comprising the blood sample/reagent mixture. Place the fluid transferring device's other end in the centre of the fluid receiving device described in example 11 above, with filter material with immobilised anti theophylline antibodies described in example 8 above. The sample/reagent mixture is allowed to flow through the fluid transferring device into the said fluid receiving device. The theophylline present in solution reacts much faster with the antibodies immobilised in the porous material than the conjugates on the microspheres, and compete efficiently with the binding of the blue microspheres in the filter material. When the assay solution has been depleted from free theophylline in solution in the migration process, the blue microspheres, with 8-(3-carboxypropyl)-1,3-dimethylxanthine conjugate with bovine serum albumin bound to the blue microspheres, bind without the said competition with the immobilised monoclonal anti-theophylline antibodies, and produces a more fluorescent ring outside the inner less dense area. When the porous filter material in the fluid receiving device has been saturated with liquid, the flow of liquid through the fluid transferring device stops automatically.

The fluid receiving device is scanned with a fluorescence scanner with excitation wavelength of 648 nm, and the fluorescence of the cyanine-5 the surface of the fluid receiving device is measured. By software computing according to example 16 above and as described by Bremnes and Sundrehagen, the area of the less fluorescence is depicted and measured. To establish a calibrated reliable quantitative method in the wanted concentration range of theophylline in whole blood, use calibrators of human whole blood with known concentrations of theophylline.

1. A quantitative chemical method of analysis for determining concentrations of one or several analytes in a sample, characterized in that a sample containing the analyte or analytes is mixed with a reagent contained in a container, wherein the reagent contains signal-providing substance(s), thus providing a mixture which is subsequently absorbed by a fluid-transmitting material contained in a fluid-transmitting device after coupling of the container to the fluid-transmitting device, and simultaneously or afterwards bringing the fluid-transmitting device in contact with a fluid-receiving device containing a fluid-receiving material which includes immobilized reagents with specific binding capacity for the analyte or analytes, or immobilized analyte molecules or analogues or derivates or fragments thereof, wherein the mixture is transported out in the porous fluid-receiving material in the said other fluid-receiving device and create a pattern wherein the pattern or area of the pattern or area of the pattern elements are utilized as a measure of the concentration of analyte or analytes in the sample.

2. A quantitative chemical method of analysis for determining concentrations of one or several analytes in a test sample according to claim 1, characterized by the following steps;
the sample is mixed with the reagent, such as a liquid reagent in a container containing signal-providing substances,
a fluid-transmitting device containing a fluid-transmitting material is introduced into the said container so that the said fluid-transmitting material comes into contact with the said mixture in the said container,
the said fluid-transmitting material in the said fluid-transmitting device in the course of performing the said chemical method of analysis is brought into simultaneous contact with on the one hand the said mixture of reagent and test sample and on the other hand into contact with a porous fluid-receiving material in another fluid-receiving device, wherein the said fluid-transmitting material in the said fluid-transmitting device is not permanently mounted in contact with the porous fluid-receiving material in the said fluid-receiving device, but is brought into such contact as a part of performing this method, and wherein the said porous fluid-receiving material in the said other fluid-receiving device includes immobilized reagents which have specific binding affinity for the said analyte or analytes or that the said immobilized reagents consist of immobilized analyte molecules or analogues or derivatives or fragments of analyte molecules,
whereby the said mixture is transported through the fluid-transmitting device and over into and spreads out in the porous fluid-receiving material in the said other fluid-receiving device, whereby the pattern, the area of the pattern and/or the area of the pattern elements that emerge through the distribution of the signal-providing substances in the said porous fluid-receiving material in the said fluid-receiving device, are utilized as a measure of the concentration of analyte or analytes in the sample.

3. A quantitative chemical method of analysis for determining concentrations of one or several analytes in a sample in accordance with claim 1, characterized by the said container being a liquid leak proof container, and [further characterized by] the fluid-transmitting device, which contains a fluid-transmitting material, being led through a liquid leak proof gate into the said container in such a way that the said fluid-transmitting material comes into contact with the said mixture in the said container.

4. A quantitative chemical method of analysis for determining concentrations of one or several analytes in a sample in accordance with claim 1-3, characterized (by the fact) in that the fluid-transporting material in the fluid-transmitting device consists of a porous fluid-transporting material suitable for transporting fluids using capillary forces or overpressure or underpressure.

5. A quantitative chemical method of analysis for determining concentrations of one or several analytes in a sample in accordance with one or several of the claims 1 to 4, characterized by the inclusion in the fluid transmission device of a non-porous nib or a tube-shaped transmission which is not mounted in permanent contact with the fluid-receiving device, but which is brought into contact with the fluid-receiving device during the process of carrying out the quantitative chemical method of analysis.

6. A quantitative chemical method of analysis for determining concentrations of one or several analytes in a sample in accordance with one or several of the claims 1 to 5, characterized (by the fact) in that the said container for mixing of reagent with test sample is a closed container with a gate at which the said fluid-transmitting device can come into contact with the said mixture, (if expedient) suitably by supplying the said container with a notch in a wall where the wall is thinner and yields when the transmission device is led through in a tight fitting manner or, (if expedient) by the fluid-transmitting unit and the said container being screwed together, (if expedient) suitably with small gas-permeable openings in the container or transmission device, shaped in such a manner that the said mixture does not leak out of the container or fluid transmission device regardless of the spatial position in which the container (and/or) with the fluid transmission device is held.

7. A quantitative chemical method of analysis for determining concentrations of one or several analytes in a sample in accordance with one or several of the claims 1 to 6, characterized (by the fact) in that the said container for mixing of reagent and test sample is equipped with a (gate) port for introduction of the test sample or that a third device containing the test sample is used and, if desirable, that the said third device constitutes a part of the said container when it is joined together with or screwed onto the other devices.

8. A quantitative chemical method of analysis for determining concentrations of one or several analytes in a sample in accordance with claim 7, characterizedin that the said third device is not a part of the container and is e.g. a glass capillary.

9. A quantitative chemical method of analysis for determining concentrations of one or several analytes in a sample in accordance with one or several of the claims 1 to 8, characterized by the said fluid-receiving device containing specific binding molecules with affinity for analytes or the analytes, or for analogues of or derivatives of or fragments of or whole analyte molecules, either in immobilized form and/or in desiccated form or dispersed onto or into particles or directly into the porous fluid-receiving material in the said fluid-receiving device, with a homogeneous or inhomogeneous, but previously determined, distribution in the porous fluid-receiving material.

10. A quantitative chemical method of analysis for determining concentrations of one or several analytes in a sample in accordance with one or several of the claims 1 to 9, characterized by the said reagent containing signal-providing substances in the form of colored particles or colloids or enzymes or fluorophores or dyes, with or without attached specific binding molecules or with or without attached analogues of or derivatives of or fragments of or whole analyte molecules.

11. A quantitative chemical method of analysis for determining concentrations of one or several analytes in a sample in accordance with one or several of the claims 1 to 10, characterized by the said reagent including chemicals that dissolve cells in the test sample and/or regulate the acidity or ionic strength or keep any possible particles dispersed.

12. A quantitative chemical method of analysis for determining concentrations of one or several analytes in a sample in accordance with one or several of the claims 1 to 11, characterized by the said fluid-transmitting device having a pore size that holds back cells such as red or white blood cells, but with a pore size large enough to let through the said signal-providing substances.

13. A quantitative chemical method of analysis for determining concentrations of one or several analytes in a sample in accordance with one or several of the claims 1 to 12, characterized (by) in that the hemoglobin in the test sample being used as signal-providing substance.

14. A quantitative chemical method of analysis for determining concentrations of one or several analytes in a sample in accordance with one or several of the claims 1 to 13, characterized by the test sample being pretreated by adding chemicals or separated or extracted prior to being mixed with the said reagent or that the said reagent can be provided by mixing together two or several different reagents inside the said container, or that additional chemicals are admixed into the porous fluid-receiving material in the fluid-receiving device in order to evoke or enhance or clarify the patterns or areas of patterns and/or the area of the pattern elements that appear in the said fluid-receiving device.

15. A quantitative chemical method of analysis for determining concentrations of one or several analytes in a sample in accordance with one or several of the claims 1 to 14, characterized (by) in that the patterns or areas of patterns and/or the area of pattern elements that appear in the said fluid-receiving device being depicted or scanned or measured using analogue or digital instruments based on visible or ultraviolet or infrared or near-infrared light, either by absorption measurement or reflection measurement or fluorescence measurement, and that the concentration of the analyte or the analytes in the test sample is determined on the basis of these measurements.

16. A quantitative chemical method of analysis for determining concentrations of one or several analytes in a sample in accordance with one or several of the claims 1 to 15, characterized by the use of a leak proof container for mixing reagent and test sample, and further characterized by the fact that the fluid-transmitting device contains a porous fluid-transmitting material which is mounted in permanent contact with the porous fluid-receiving material in the fluid-receiving device.

17. A quantitative chemical method of analysis for determining concentrations of one or several analytes in a sample in accordance with one or several of the claims 1 to 16, characterized in that the test sample is a biological sample.

18. A device for performing a method for determining concentrations of one or several analytes in a test sample, characterized in comprising a liquid leak proof container for mixing of the test sample with a reagent, a fluid-transmitting device which contains a fluid-transmitting material, and a fluid-receiving device which contains a fluid-receiving material, assembled such that the fluid-transmitting device is able to be contacted with the content of the said container through a liquid leak proof port and contacted with the fluid-receiving device containing the fluid-receiving material.

19. A device in accordance with claim 18, characterized in that the fluid-device transporting material in the fluid-transmitting device consists of a porous fluid-transporting material suitable for transporting fluids using capillary forces or overpressure or underpressure.

20. A device in accordance with claims 18 to 19, characterized in that a non-porous nib or a tube-shaped transmission is included in the fluid transmission device, not mounted in permanent contact with the fluid-receiving device, but brought into contact with the fluid-receiving device during the process of carrying out the quantitative chemical method of analysis.

21. A device in accordance with claims 18 to 20, characterized in that the said leak proof container has a port through which the said fluid-transmitting device can come into contact with the said mixture of test sample and reagent, suitably that the said container has a notch in a wall where the wall is thinner and yields when the transmission device is led through in a tight fitting manner or the fluid-transmitting unit, and the said container being screwed together, suitably with small gas-permeable openings in the container or transmission, shaped in such a manner that the said mixture does not leak out of the container or fluid transmission device regardless of the spatial position in which the container with the fluid transmission device is held.

22. A device in accordance with claims 18 to 21, characterized in that the said container for mixing of reagent and test sample is equipped with a port for introduction of the test sample from a sample transporting device, such as e.g. a glass capillary, or that the sample transporting device constitutes a part of the said container, such as a lid device which is joined together with, or screwed onto the said container in the port location.

23. A device in accordance with claims 18 to 22, characterized by the said fluid-receiving device containing specific binding molecules with affinity for analytes or the analytes, or for analogues of or derivatives of or fragments of or whole analyte molecules, either in immobilized form and/or in desiccated form or dispersed onto or into particles or directly into the porous fluid-receiving material in the said fluid-receiving device, with a homogeneous or inhomogeneous, but previously determined., distribution in the porous fluid-receiving material.

24. A device in accordance with claims 18 to 23, characterized in that the said container contained reagent comprises signal-providing substances in the form of colored particles or colloids or enzymes or fluorophores or dyes, with or without attached specific binding molecules or with or without attached analogues of or derivatives of or fragments of or whole analyte molecules.

25. A device in accordance with claims 18 to 24, characterized in that the said reagent includes chemicals that dissolve cells in the test sample and/or regulate the acidity or ionic strength or keep any possible particles dispersed.

26. A device in accordance with claims 18 to 25, characterized in that the said fluid-transmitting material in the said fluid-transmitting device has a pore size that holds back cells, such as red or white blood cells, but with a pore size large enough to let through the said signal-providing substances.

27. A device in accordance with claims 18 to 26, characterized in comprising a stopper (6) with a built in capillary (7), a sealing sleeve (8) surrounding the stopper, a liquid leak proof container (9), a movable ball (10) sealing the port in the bottom of the container (9), wherein the ball (10) is housed in a valve seat(11) which is sealingly fitted to a wick or felt tip guide (12), wherein a wick or felt tip (13) is sealingly and movable mounted, wherein the felt tip (13) is protected by a removable cap (14)

28. A device in accordance with claims 18 to 26, characterized by further comprising a scanning device, such as analogue or digital instrument based on visible or ultraviolet or infrared or near infrared light, or a combination thereof, to measure absorption or reflection or fluorescence, or a combination thereof, a processor for processing the data, a display medium, and medium for storing the data.

29. A device in accordance with claims 18 to 28, characterized by further comprising a rack with a movable holder, whereby the container is fixed in a standardized position in relation to the fluid-receiving device such that only vertical controlled movement is possible.

30. Use of the method according to claims 1-17 wherein the concentration of one or several analytes in a biological sample, such as blood, sputum, mucus, faeces, expectorates and tissue is measured.

31. Use of the method according to claim 1, wherein the analytes are selected from the group comprising autoantibodies, antibodies, saprophytes, bacteria, other infectious agents, hemoglobin, albumin, CRP, U-albumin, glycated albumin, glycated hemoglobin, ferritin, ASAT, ALAT, LDH, myoglobin, Troponin I, Fatty Acid Binding Protein, amylase, HCG, U-HCG, theophyllin, and antibiotics.

32. Kit for performing the method according to claims 1-17, characterized in comprising the device according to claims 18-31, reagent for mixing with the test sample, optionally additional reagents for pretreatment or separation of the test sample or admixing into the fluid-receiving device for clarification of the signal.
